(19) United States
(12) Patent Application Publication
(10) Pub. No.: US 2004/0115268 A1
(43) Pub. Date: Jun. 17, 2004

(54) Systemic delivery of antiviral agents

(75) Inventors: Paul Ashton, Boston, MA (US); Jianbing Chen, Belmont, MA (US); Thomas J. Smith, Weston, MA (US)

Correspondence Address: ROPES & GRAY LLP, ONE INTERNATIONAL PLACE, BOSTON, MA, 02110-2624, US

(73) Assignee: Control Delivery Systems, Inc., Watertown, MA

(21) Appl. No.: 10/713,336

(22) Filed: Nov. 13, 2003

Related U.S. Application Data
(63) Continuation-in-part of application No. 10/096,877, filed on Mar. 14, 2002.
Application No. 10/096,877 is a continuation of application No. 09/558,207, filed on Apr. 26, 2000, now Pat. No. 6,375,972.
(60) Provisional application No. 60/425,943, filed on Nov. 13, 2002.

(51) Int. Cl. A61K 9/24

(52) U.S. Cl. 424/473

(57) ABSTRACT

The systems and methods disclosed herein provide sustained delivery of a therapeutic agent for treating a patient, e.g., human, to obtain a desired local or systemic physiological or pharmacological effect. Method includes positioning the sustained released drug delivery system at an area wherein release of the agent is desired and allowing the agent to pass through the device to the desired area of treatment. In some embodiments, the method is for treating or reducing the risk of retroviral or lentiviral infection. In certain embodiments, the method is for preventing or reducing the risk of mother-to-child transmission of HIV, wherein the therapeutic agent is an antiviral agent.

### RELATED APPLICATIONS

This application is a continuation in part of U.S. application Ser. No. 10/096,877, filed Mar. 14, 2002, which is a continuation of U.S. Pat. No. 6,375,972, filed Apr. 26, 2000. This application also claims the benefit of U.S. Application No. 60/425,943, filed Nov. 13, 2002. The specifications of each of the above are incorporated by reference herein.

### BACKGROUND OF THE INVENTION

The type-1 human immunodeficiency virus (HIV-1) has been implicated as the primary cause of the degenerative disease of the immune system termed acquired immune deficiency syndrome (AIDS) (Barr-Sinoussi, F. et al., 1983 Science 220:868-70; Gallo, R. et al. 1984, Science 224:500-3). Infection of the CD4+subclass of T-lymphocytes with the HIV-1 virus leads to depletion of this essential lymphocyte subclass which inevitably leads to opportunistic infections, neurological disease, neoplastic growth and eventually death. HIV-1 infection and HIV-1 associated diseases represent a major health problem and considerable attention is currently being directed towards the successful design of effective therapeutics.

HIV-1 is a member of the lentivirus family of retroviruses (Teich, N. et al., 1984 In RNA Tumor Viruses ed. R. Weiss, N. Teich, H. Varmus, J. Coffin CSH Press, pp. 949-56). The life cycle of HIV-1 is characterized by a period of proviral latency followed by active replication of the virus. The primary cellular target for the infectious HIV-1 virus is the CD4⁺ subset of human T-lymphocytes. Targeting of the virus to the CD4⁺ subset of cells is due to the fact that the CD4⁺ cell surface protein acts as the cellular receptor for the HIV-1 virus (Dalgleish, A. et al., 1984, Nature 312:763-67; Klatzmann et al. 1984, Nature 312:767-68; Maddon et al. 1986 Cell 47:333-48).

Almost all HIV-infected children acquire the virus from their mothers before or during birth or through breast-feeding. In the United States, approximately 25 percent of pregnant HIV-infected women not receiving AZT therapy pass on the virus to their babies. The rate is higher in developing countries.

Most mother-to-child transmission, estimated to cause over 90 percent of infections worldwide in infants and children, probably occurs late in pregnancy or during birth. Although the precise mechanisms are unknown, scientists think HIV may be transmitted when maternal blood enters the fetal circulation, or by mucosal exposure to virus during labor and delivery. The role of the placenta in maternal-fetal transmission is unclear and the focus of ongoing research.

The risk of maternal-infant transmission is significantly increased if the mother has advanced HIV disease, increased levels of HIV in her bloodstream, or fewer numbers of the immune system cells—CD4⁺ T cells—that are the main targets of HIV.

HIV also may be transmitted from a nursing mother to her infant. Studies have suggested that breast-feeding introduces an additional risk of HIV transmission of approximately 10 to 14 percent among women with chronic HIV infection. In developing countries, an estimated one-third to one-half of all HIV infections are transmitted through breast-feeding. The World Health Organization recommends that all HIV-infected women be advised as to both the risks and benefits of breast-feeding of their infants so that they can make informed decisions. In countries where safe alternatives to breast-feeding are readily available and economically feasible, this alternative should be encouraged. In general, in developing countries where safe alternatives to breast-feeding are not readily available, the benefits of breast-feeding in terms of decreased illness and death due to other infectious diseases greatly outweigh the potential risk of HIV transmission.

### SUMMARY OF THE INVENTION

One embodiment of the present invention provides a device suitable for the controlled and sustained release of an antiviral composition effective in obtaining a desired local or systemic physiological or pharmacological effect.

Another embodiment provides a method for treating a patient, e.g., human, to obtain a desired local or systemic physiological or pharmacological effect. The method includes positioning the sustained released drug delivery system at an area wherein release of the agent is desired and allowing the agent to pass through the device to the desired area of treatment. In some embodiments, the method is for treating or reducing the risk of retroviral or lentiviral infection. In certain embodiments, the method is for preventing or reducing the risk of mother-to-child transmission of HIV, wherein the therapeutic agent is an antiviral agent.

The drug delivery systems of the present invention may be inserted into intradermal, intramuscular, intraperitoneal, or subcutaneous sites. Insertion may be achieved by injecting the system, surgically implanting the system, or otherwise administering the system.

According to an exemplary embodiment, a sustained release drug delivery system comprises an inner reservoir comprising a therapeutically effective amount of an antiviral agent, an inner tube impermeable to the passage of said agent, said inner tube having first and second ends and covering at least a portion of said inner reservoir, said inner tube being dimensionally stable, an impermeable member positioned at said inner tube first end, said impermeable member preventing passage of said agent out of said reservoir through said inner tube first end, and a permeable member positioned at said inner tube second end, said permeable member allowing diffusion of said agent out of said reservoir through said inner tube second end.

According to another exemplary embodiment, a sustained release drug delivery system comprises a drug core comprising a therapeutically effective amount of an antiviral agent, a first polymer coating permeable to the passage of said agent, and

a second polymer coating impermeable to the passage of said agent, wherein the second polymer coating covers a portion of the surface area of the drug core and/or the first polymer coating.

According to another embodiment, a method for providing controlled and sustained administration of an agent effective in obtaining a desired local or systemic physiological or pharmacological effect comprises surgically implanting a sustained release drug delivery system at a desired location.

According to yet another embodiment, a method of manufacturing a sustained release drug delivery system comprises manufacturing a drug core, coating the drug core with a permeable polymer, and encasing the coated drug core in an impermeable tube.

Still other features of the present invention will become apparent to those skilled in the art from a reading of the following detailed description of embodiments constructed in accordance therewith, taken in conjunction with the accompanying drawings.

### BRIEF DESCRIPTION OF THE FIGURES

The invention of the present application will now be described in more detail with reference to preferred embodiments of the apparatus and method, given only by way of example, and with reference to the accompanying drawings, in which:

FIG. 1 is an enlarged cross-sectional illustration of one embodiment of a sustained release drug delivery device in accordance with the present invention;

FIG. 2 is an enlarged cross-sectional illustration of a second embodiment of a sustained release drug delivery device in accordance with the present invention;

FIG. 3 is an enlarged cross-sectional illustration of a third embodiment of a sustained release drug delivery device in accordance with the present invention;

FIG. 4 is a cross-sectional illustration of the embodiment illustrated in FIG. 2, taken at line 4-4;

FIG. 5 schematically illustrates an embodiment of a method in accordance with the present invention of fabricating a drug delivery device;

FIG. 6 is a graph showing the release profile of nevirapine, expressed as cumulative release, from a sustained release drug delivery device in accordance with the present invention;

FIG. 7 is a graph showing the concentration of nevirapine in rat plasma over a period of more than 90 days from six sustained release drug delivery devices in accordance with the present invention;

FIG. 8 is a graph showing the release profile of nevirapine, expressed as cumulative release, from a sustained release drug device in accordance with the present invention;

FIG. 9 is a graph showing the concentration of nevirapine in rat plasma over a period of more than 90 days from a sustained release drug delivery device in accordance with the present invention;

FIG. 10 is a graph showing the concentration of nevirapine in rat plasma over a period of more than 90 days from one sustained release drug delivery device in accordance with the present invention;

FIG. 11 is a graph showing the release profile of nevirapine, expressed as cumulative release, from a sustained release drug delivery device in accordance with the present invention; and

FIG. 12 is a graph showing the concentration of nevirapine in rat plasma over a period of more than 90 days from a sustained release drug delivery device in accordance with the present invention.

### DETAILED DESCRIPTION OF THE INVENTION

The present invention provides for sustained release formulations and devices for systemic delivery of antiviral agents. In preferred embodiments, the subject invention provides methods and compositions for treating or reducing the risk of retroviral or lentiviral infection, such as in the treatment of HIV.

The present invention particularly contemplates sustained release compositions for systemic delivery of an antiviral drug that can protect infants from mother-to-child transmission of viral infections, e.g., to protect infants from maternal transmission of HIV, especially as a consequence of nursing.

In certain embodiments, the antiviral agent(s) are prepared for sustained release from intradermal, intramuscular, intraperitoneal, or subcutaneous sites. For instance, the antiviral agent can be formulated in a polymer or hydrogel which can be introduced at site in the body where it remains reasonably dimensionally stable and localized for at least a period of days, and more preferably for 2-10 weeks or more. In other embodiments, the antiviral agent can be provided in a sustained release device, which in turn can implanted at a position in the body, preferably where (or by means of securing the device) it is not likely to migrate—at least not from the compartment in which it is implanted.

One aspect of the invention provides a sustained release drug delivery system comprising an inner drug core comprising an amount of an antiviral agent, an inner tube impermeable to the passage of said agent, said inner tube having first and second ends and covering at least a portion of said inner drug core, said inner tube being dimensionally stable, an impermeable member positioned at said inner tube first end, said impermeable member preventing passage of said agent out of said drug core through said inner tube first end, and a permeable member positioned at said inner tube second end, said permeable member allowing diffusion of said agent from said drug core through said inner tube second end.

Another aspect of the invention provides a sustained release drug delivery system comprising a drug core comprising an amount of an antiviral agent, a first polymer coating permeable to the passage of said agent, and a second polymer coating impermeable to the passage of said agent, wherein the second polymer coating covers a portion of the surface area of the drug core and/or the first polymer coating.

Another aspect of the invention provides a sustained release drug delivery system comprising a drug core comprising an amount of an antiviral agent, a first polymer coating and a second polymer coating permeable to the passage of said agent, wherein the two polymer coatings are bioerodible and erode at different rates.

A further aspect of the invention provides a sustained release drug delivery system comprising a drug core comprising an amount of an antiviral agent, a first polymer coating permeable to the passage of said agent covering at least a portion of the drug core, a second polymer coating essentially impermeable to the passage of said agent covering at least a portion of the drug core and/or the first polymer coating, and a third polymer coating permeable to the passage of said agent essentially completely covering the drug core and the second polymer coating, wherein a dose of said agent is released for at least 7 days.

Another aspect of the invention provides a sustained release drug delivery system comprising a drug core comprising an amount of an antiviral agent, a first polymer coating permeable to the passage of said agent covering at least a portion of the drug core, a second polymer coating essentially impermeable to the passage of said agent covering at least a portion of the drug core and/or the first polymer coating, and a third polymer coating permeable to the passage of said agent essentially completely covering the drug core and the second polymer coating, wherein release of said agent maintains a desired concentration of said agent in blood plasma for at least 7 days.

Yet still another aspect of the invention provides a sustained release drug delivery system comprising a drug core comprising an amount of an antiviral agent, and a non-erodible polymer coating, the polymer coating being permeable to the passage of said agent covering the drug core and is essentially non-release rate limiting, wherein a dose of said agent is released for at least 7 days.

A further aspect of the invention provides a sustained release drug delivery system comprising a drug core comprising an amount of an antiviral agent, and a non-erodible polymer coating, the polymer coating being permeable to the passage of said agent covering the drug core and being essentially non-release rate limiting, wherein release of said agent maintains a desired concentration of said agent in blood plasma for at least 7 days.

Another aspect of the invention provides a sustained release drug delivery system comprising a drug core comprising an amount of an antiviral agent, a first polymer coating permeable to the passage of said agent covering at least a portion of the drug core, a second polymer coating essentially impermeable to the passage of said agent covering at least 50% of the drug core and/or the first polymer coating, said second polymer coating comprising an impermeable film and at least one impermeable disc, and a third polymer coating permeable to the passage of said agent essentially completely covering the drug core, the uncoated portion of the first polymer coating, and the second polymer coating, wherein an dose of said agent is released for at least 7 days.

Another aspect of the invention provides a sustained release drug delivery system comprising a drug core comprising an amount of an antiviral agent, a first polymer coating permeable to the passage of said agent covering at least a portion of the drug core, a second polymer coating essentially impermeable to the passage of said agent covering at least 50% of the drug core and/or the first polymer coating, said second polymer coating comprising an impermeable film and at least one impermeable disc, and a third polymer coating permeable to the passage of said agent essentially completely covering the drug core, the uncoated portion of the first polymer coating, and the second polymer coating, wherein release of said agent maintains a desired concentration of said agent in blood plasma for at least 7 days.

Yet still another aspect of the invention provides a method for treating or reducing the risk of retroviral or lentiviral infection comprising implanting a sustained release drug delivery system including an antiviral agent in a patient in need of treatment wherein a dose of said agent is released for at least 7 days.

Another aspect of the invention provides a method for treating or reducing the risk of retroviral or lentiviral infection comprising implanting a sustained release drug delivery system including an antiviral agent in a patient in need of treatment wherein release of said agent maintains a desired concentration of said agent in blood plasma for at least 7 days.

In certain embodiments, the system reduces the risk of mother to child transmission of viral infections. Examples of viral infections include HIV, Bowenoid Papulosis, Chickenpox, Childhood HIV Disease, Human Cowpox, Hepatitis C, Dengue, Enteroviral, Epidermodysplasia Verruciformis, Erythema Infectiosum (Fifth Disease), Giant Condylomata Acuminata of Buschke and Lowenstein, Hand-Foot-and-Mouth Disease, Herpes Simplex, Herpes Virus 6, Herpes Zoster, Kaposi Varicelliform Eruption, Rubeola Measles, Milker's Nodules, Molluscum Contagiosum, Monkeypox, Orf, Roseola Infantum, Rubella, Smallpox, Viral Hemorrhagic Fevers, Genital Warts, and Nongenital Warts.

In some embodiments, the antiviral agent is selected from azidouridine, anasmycin, amantadine, bromovinyldeoxusidine, chlorovinyldeoxusidine, cytarbine, didanosine, deoxynojirimycin, dideoxycitidine, dideoxyinosine, dideoxynucleoside, desciclovir, deoxyacyclovir, edoxuidine, enviroxime, fiacitabine, foscamet, fialuridine, fluorothymidine, floxuridine, hypericin, interferon, interleukin, isethionate, nevirapine, pentamidine, ribavirin, rimantadine, stavirdine, sargramostin, suramin, trichosanthin, tribromothymidine, trichlorothymidine, vidarabine, zidoviridine, zalcitabine and 3-azido-3-deoxythymidine, and analogs, derivatives, pharmaceutically acceptable salts, esters, prodrugs, codrugs, and protected forms thereof. In certain embodiments, the antiviral agent is selected from nevirapine, delavirdine and efavirenz, and analogs, derivatives, pharmaceutically acceptable salts, esters, prodrugs, codrugs, and protected forms thereof. In preferred embodiments, the antiviral agent is nevirapine.

In other embodiments, the antiviral agent is selected from 2′,3′-dideoxyadenosine (ddA), 2′,3′-dideoxyguanosine (ddG), 2′,3′-dideoxycytidine (ddC), 2′,3′-dideoxythymidine (ddT), 2′3′-dideoxy-dideoxythymidine (d4T), 2′-deoxy-3′-thia-cytosine (3TC or lamivudime), 2′,3′-dideoxy-2′-fluoroadenosine, 2′,3′-dideoxy-2′-fluoroinosine, 2′,3′-dideoxy-2′-fluorothymidine, 2′,3′-dideoxy-2′-fluorocytosine, 2′3′-dideoxy-2′,3′-didehydro-2′-fluorothymidine (Fd4T), 2′3′-dideoxy-2′-beta-fluoroadenosine (F-ddA), 2′3′-dideoxy-2′-beta-fluoro-inosine (F-ddI), and 2′,3′-dideoxy-2′-beta-flurocytosine (F-ddC), and analogs, derivatives, pharmaceutically acceptable salts, esters, prodrugs, codrugs, and protected forms thereof.

In some embodiments, the antiviral agent is selected from trisodium phosphomonoformate, ganciclovir, trifluorothymidine, acyclovir, 3′azido-3′thymidine (AZT), dideoxyinosine (ddI), and idoxuridine, and analogs, derivatives, pharmaceutically acceptable salts, esters, prodrugs, codrugs, and protected forms thereof.

Codrugs or prodrugs may be used to deliver drugs, including antiviral agents of the present invention, in a sustained manner. In certain embodiments, codrugs and prodrugs may be adapted to use in the core or outer layers of the drug delivery devices described herein. An example of sustained-release systems using codrugs and prodrugs may be found in U.S. Pat. No. 6,051,576. This patent is incorporated in its entirety herein by reference. In other embodiments, codrugs and prodrugs may be included with the gelling, suspension, and other embodiments described herein.

As used herein, the term “codrug” means a first constituent moiety chemically linked to at least one other constituent moiety that is the same as, or different from, the first constituent moiety. The individual constituent moieties are reconstituted as the pharmaceutically active forms of the same moieties, or codrugs thereof, prior to conjugation. Constituent moieties may be linked together via reversible covalent bonds such as ester, amide, carbamate, carbonate, cyclic ketal, thioester, thioamide, thiocarbamate, thiocarbonate, xanthate and phosphate ester bonds, so that at the required site in the body they are cleaved to regenerate the active forms of the drug compounds.

As used herein, the term “constituent moiety” means one of two or more pharmaceutically active moieties so linked as to form a codrug according to the present invention as described herein. In some embodiments according to the present invention, two molecules of the same constituent moiety are combined to form a dimer (which may or may not have a plane of symmetry). In the context where the free, unconjugated form of the moiety is referred to, the term “constituent moiety” means a pharmaceutically active moiety, either before it is combined with another pharmaceutically active moiety to form a codrug, or after the codrug has been hydrolyzed to remove the linkage between the two or more constituent moieties. In such cases, the constituent moieties are chemically the same as the pharmaceutically active forms of the same moieties, or codrugs thereof, prior to conjugation.

The term “prodrug” is intended to encompass compounds that, under physiological conditions, are converted into the therapeutically active agents of the present invention. A common method for making a prodrug is to include selected moieties, such as esters, that are hydrolyzed under physiological conditions to convert the prodrug to an active biological moiety. In other embodiments, the prodrug is converted by an enzymatic activity of the host animal. Prodrugs are typically formed by chemical modification of a biologically active moiety. Conventional procedures for the selection and preparation of suitable prodrug derivatives are described, for example, in Design of Prodrugs, ed. H. Bundgaard, Elsevier, 1985.

In the context of referring to the codrug according to the present invention, the term “residue of a constituent moiety” means that part of a codrug that is structurally derived from a constituent moiety apart from the functional group through which the moiety is linked to another constituent moiety. For instance, where the functional group is —NH₂, and the constituent group forms an amide (—NH—CO—) bond with another constituent moiety, the residue of the constituent moiety is that part of the constituent moiety that includes the —NH— of the amide, but excluding the hydrogen (H) that is lost when the amide bond is formed. In this sense, the term “residue” as used herein is analogous to the sense of the word “residue” as used in peptide and protein chemistry to refer to a residue of an amino acid in a peptide.

Codrugs may be formed from two or more constituent moieties covalently linked together either directly or through a linking group. The covalent bonds between residues include a bonding structure such as:

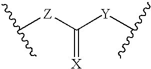

wherein Z is O, N, —CH₂—, —CH₂—O— or —CH₂—S—, Y is O, or N, and X is O or S. The rate of cleavage of the individual constituent moieties can be controlled by the type of bond, the choice of constituent moieties, and/or the physical form of the codrug. The lability of the selected bond type may be enzyme-specific. In some embodiments, the bond is selectively labile in the presence of an esterase. In other embodiments of the invention, the bond is chemically labile, e.g., to acid- or base-catalyzed hydrolysis. In some embodiments, the linking group does not include a sugar, a reduced sugar, a pyrophosphate, or a phosphate group.

The physiologically labile linkage may be any linkage that is labile under conditions approximating those found in physiologic fluids. The linkage may be a direct bond (for instance, ester, amide, carbamate, carbonate, cyclic ketal, thioester, thioamide, thiocarbamate, thiocarbonate, xanthate, phosphate ester, sulfonate, or a sulfamate linkage) or may be a linking group (for instance, a C₁-C₁₂ dialcohol, a C₁-C₁₂ hydroxyalkanoic acid, a C₁-C₁₂ hydroxyalkylamine, a C₁-C₁₂ diacid, a C₁-C₁₂ aminoacid, or a C₁-C₁₂ diamine). Especially preferred linkages are direct amide, ester, carbonate, carbamate, and sulfamate linkages, and linkages via succinic acid, salicylic acid, diglycolic acid, oxa acids, oxamethylene, and halides thereof. The linkages are labile under physiologic conditions, which generally means pH of about 6 to about 8. The lability of the linkages depends upon the particular type of linkage, the precise pH and ionic strength of the physiologic fluid, and the presence or absence of enzymes that tend to catalyze hydrolysis reactions in vivo. In general, lability of the linkage in vivo is measured relative to the stability of the linkage when the codrug has not been solubilized in a physiologic fluid. Thus, while some codrugs may be relatively stable in some physiologic fluids, nonetheless, they are relatively vulnerable to hydrolysis in vivo (or in vitro, when dissolved in physiologic fluids, whether naturally occurring or simulated) as compared to when they are neat or dissolved in non-physiologic fluids (e.g., non-aqueous solvents such as acetone). Thus, the labile linkages are such that, when the codrug is dissolved in an aqueous solution, the reaction is driven to the hydrolysis products, which include the constituent moieties set forth above.

Codrugs for preparation of a drug delivery device for use with the systems described herein may be synthesized in the manner illustrated in one of the synthetic schemes below. In general, where the first and second constituent moieties are to be directly linked, the first moiety is condensed with the second moiety under conditions suitable for forming a linkage that is labile under physiologic conditions. In some cases it is necessary to block some reactive groups on one, the other, or both of the moieties. Where the constituent moieties are to be covalently linked via a linker, such as oxamethylene, succinic acid, or diglycolic acid, it is advantageous to first condense the first constituent moiety with the linker. In some cases it is advantageous to perform the reaction in a suitable solvent, such as acetonitrile, in the presence of suitable catalysts, such as carbodiimides including EDCI (1-ethyl-3-(3-dimethylaminopropyl)-carbodiimide) and DCC (DCC:dicyclohexylcarbo-diimide), or under conditions suitable to drive off water of condensation or other reaction products (e.g., reflux or molecular sieves), or a combination of two or more thereof. After the first constituent moiety is condensed with the linker, the combined first constituent moiety and linker may then be condensed with the second constituent moiety. Again, in some cases it is advantageous to perform the reaction in a suitable solvent, such as acetonitrile, in the presence of suitable catalysts, such as carbodiimides including EDCI and DCC, or under conditions suitable to drive off water of condensation or other reaction products (e.g., reflux or molecular sieves), or a combination of two or more thereof. Where one or more active groups have been blocked, it may be advantageous to remove the blocking groups under selective conditions, however it may also be advantageous, where the hydrolysis product of the blocking group and the blocked group is physiologically benign, to leave the active groups blocked.

The person having skill in the art will recognize that, while diacids, dialcohols, amino acids, etc., are described as being suitable linkers, other linkers are contemplated as being within the present invention. For instance, while the hydrolysis product of a codrug described herein may comprise a diacid, the actual reagent used to make the linkage may be, for example, an acylhalide such as succinyl chloride. The person having skill in the art will recognize that other possible acid, alcohol, amino, sulfato, and sulfamoyl derivatives may be used as reagents to make the corresponding linkage.

Where the first and second constituent moieties are to be directly linked via a covalent bond, essentially the same process is conducted, except that in this case there is no need for a step of adding a linker. The first and second constituent moieties are merely combined under conditions suitable for forming the covalent bond. In some cases it may be desirable to block certain active groups on one, the other, or both of the constituent moieties. In some cases it may be desirable to use a suitable solvent, such as acetonitrile, a catalyst suitable to form the direct bond, such as carbodiimides including EDCI and DCC, or conditions designed to drive off water of condensation (e.g., reflux) or other reaction by-products.

While in some cases the first and second moieties may be directly linked in their original form, it is possible for the active groups to be derivatized to increase their reactivity. For instance, where the first moiety is an acid and the second moiety is an alcohol (i.e., has a free hydroxyl group), the first moiety may be derivatized to form the corresponding acid halide, such as an acid chloride or an acid bromide. The person having skill in the art will recognize that other possibilities exist for increasing yield, lowering production costs, improving purity, etc., of the codrug described herein by using conventionally derivatized starting materials to make the codrugs described herein.

One constituent moiety of the codrug may be any of the antiviral drugs listed elsewhere in this specification. The other may be any drug, including, without limitation, steroids, alpha receptor agonists, beta receptor antagonists, carbonic anhydrase inhibitors, adrenergic agents, physiologically active peptides and/or proteins, antineoplastic agents, antibiotics, analgesics, anti-inflammatory agents, muscle relaxants, anti-epileptics, anti-ulcerative agents, anti-allergic agents, cardiotonics, anti-arrhythmic agents, vasodilators, antihypertensive agents, anti-diabetic agents, anti-hyperlipidemics; anticoagulants, hemolytic agents, antituberculous agents, hormones, narcotic antagonists, osteoclastic suppressants, osteogenic promoters, angiogenesis suppressors, antibacterials, non-steroidal anti-inflammatory drugs (NSAIDs), glucocorticoids or other anti-inflammatory corticosteroids,s alkaloid analgesics, such as opioid analgesics, antivirals, such as nucleoside antivirals or a non-nucleoside antivirals, anti-benign prostatic hypertrophy (BPH) agents, anti-fungal compounds, antiproliferative compounds, anti-glaucoma compounds, immunomodulatory compounds, cell transport/mobility impeding agents, cytokines pegylated agents, alpha-blockers, anti-androgens, anti-cholinergic agents, purinergic agents, dopaminergic agents, local anesthetics, vanilloids, nitrous oxide inhibitors, anti-apoptotic agents, macrophage activation inhibitors, antimetabolites, neuroprotectants, calcium channel blockers, gamma-aminobutyric acid (GABA) antagonists, alpha agonists, anti-psychotic agents, tyrosine kinase inhibitors, nucleoside compounds, and nucleotide compounds, and analogs, derivatives, pharmaceutically acceptable salts, esters, prodrugs, codrugs, and protected forms thereof.

In certain embodiments, the first and second constituent moieties are the drug; in other embodiments, they are different drugs.

The term “drug” as it is used herein is intended to encompass all agents which provide a local or systemic physiological or pharmacological effect when administered to mammals, including without limitation any specific drugs noted in the following description and analogs, derivatives, pharmaceutically acceptable salts, esters, prodrugs, codrugs, and protected forms thereof.

In certain codrug embodiments, the first constituent moiety is an antiviral agent. In certain embodiments, the first and/or second constituent moiety is nevirapine or a pharmaceutically acceptable salt, analog, prodrug or codrug thereof.

Exemplary reaction schemes according to the present invention are illustrated in Schemes 1-4, below. These Schemes can be generalized by substituting other therapeutic agents having at least one functional group that can form a covalent bond to another therapeutic agent having a similar or different functional group, either directly or indirectly through a pharmaceutically acceptable linker. The person of skill in the art will appreciate that these schemes also may be generalized by using other appropriate linkers.

Scheme 1

R₁—COOH+R₂—OH→R₁—COO—R₂═R₁-L-R₂

wherein L is an ester linker —COO—, and R₁ and R₂ are the residues of the first and second constituent moieties or pharmacological moieties, respectively.

Scheme 2

R₁—COOH+R₂—NH₂→R₁—CONH—R₂═R₁-L-R₂

wherein L is the amide linker —CONH—, and R₁ and R₂ have the meanings given above.

Scheme 3

Step 1: R₁—COOH+HO-L-CO-Prot→R₁-COO-L-CO-Prot

wherein Prot is a suitable reversible protecting group.

Step 2: R₁—COO-L-CO-Prot→R₁—COO-L-COOH

Step 3: R₁—COO-L-COOH+R₂—OH l R₁—COO-L-COOR₂

wherein R₁, L, and R₂ have the meanings set forth above.

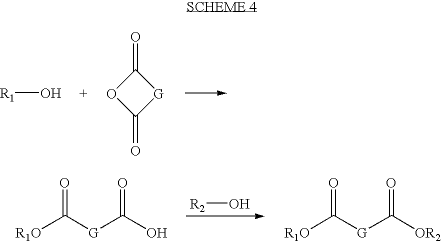

wherein R₁ and R₂ have the meanings set forth above and G is a direct bond, an C₁-C₄ alkylene, a C₂-C₄ alkenylene, a C₂-C₄ alkynylene, or a 1,2-fused ring, and G together with the anhydride group completes a cyclic anhydride. Suitable anhydrides include succinic anhydride, glutaric anhydride, maleic anhydride, diglycolic anhydride, and phthalic anhydride.

In certain embodiments, the release of the antiviral agent has a systemic effect. In other embodiments, the release of said agent has a local effect.

The amount or dose of agent released from the drug delivery systems may be a therapeutically effective or a sub-therapeutically effective amount.

In some embodiments, the amount of the agent within the drug core or reservoir is at least 1 mg to about 500 mg, preferably at least about 10 mg, 30 mg, or 50 mg. In other embodiments, the amount of the agent within the drug core or reservoir is at least about 2 mg to about 15 mg, about 15 mg to about 100 mg.

In certain embodiments, a therapeutically effective amount or dose of the agent is released for at least two weeks, one month, two months, three months, 6 months, or one year.

In some embodiments, a therapeutically effective dose is at least about 30 ng/day, 100 ng/day, or 100 μg/day. In certain embodiments, the desired concentration of said agent in blood plasma is about 20-100 ng/ml, about 40-100 ng/ml, or 60-80 ng/ml.

In certain embodiments, the system is between about 1 to 30 mm in length, preferably about 3 mm, about 5 mm, about 7 mm, or about 10 mm. In certain embodiments, the system is between about 0.5 to 5 mm in diameter, preferably about 1 mm, about 2.5 mm, or about 4 mm.

In some embodiments, the permeable member comprises a material selected from cross-linked polyvinyl alcohol, polyolefins, polyvinyl chlorides, cross-linked gelatins, insoluble and nonerodible cellulose, acylated cellulose, esterified celluloses, cellulose acetate propionate, cellulose acetate butyrate, cellulose acetate phthalate, cellulose acetate diethyl-aminoacetate, polyurethanes, polycarbonates, and microporous polymers formed by co-precipitation of a polycation and a polyanion modified insoluble collagen. In preferred embodiments, the permeable member comprises cross-linked polyvinyl alcohol.

In certain embodiments, the impermeable member comprises a material selected from polyvinyl acetate, cross-linked polyvinyl butyrate, ethylene ethyl acrylate copolymer, polyethyl hexylacrylate, polyvinyl chloride, polyvinyl acetals, plasticized ethylene vinylacetate copolymer, polyvinyl acetate, ethylene vinylchloride copolymer, polyvinyl esters, polyvinylbutyrate, polyvinylformal, polyamides, polymethylmethacrylate, polybutylmethacrylate, plasticized polyvinyl chloride, plasticized nylon, plasticized soft nylon, plasticized polyethylene terephthalate, natural rubber, polyisoprene, polyisobutylene, polybutadiene, polyethylene, polytetrafluoroethylene, polyvinylidene chloride, polyacrylonitrile, cross-linked polyvinylpyrrolidone, polytrifluorochloroethylene, chlorinated polyethylene, poly(1,4′-isopropylidene diphenylene carbonate), vinylidene chloride, acrylonitrile copolymer, vinyl chloride-diethyl fumarate copolymer, silicone rubbers, medical grade polydimethylsiloxanes, ethylene-propylene rubber, silicone-carbonate copolymers, vinylidene chloride-vinyl chloride copolymer, vinyl chloride-acrylonitrile copolymer and vinylidene chloride-acrylonitride copolymer. In some embodiments, the impermeable member comprises silicone.

In some embodiments, the impermeable member is a tube.

In certain embodiments, the second polymer coating is a dimensionally stable tube. In some embodiments, the dimensionally stable tube includes one or more pores, for example, along the surface of the tube, to achieve the desired amount of drug released. The shape of a pore is not limited to any particular shape but may be in the shape of a slit, a circular hole, or any other geometrical shape.

In some embodiments, the drug core comprises a pharmaceutically acceptable carrier. In certain embodiments, the drug core comprises 0.1 to 100% drug. In one embodiment, the drug core comprises 0.1 to 100% drug, 0.1 to 10% magnesium stearate, and 0.1 to 10% polyethylene glycol.

Another aspect of the invention provides a pharmaceutical package including one or more antiviral compounds formulated for sustained release (such as in a sustained release device), and associated with instructions or a label for use in infants who are nursing or otherwise at risk of maternal transmission of virus.

Exemplary antiviral drugs include acyclovir, azidouridine, anasmycin, amantadine, bromovinyldeoxusidine, chlorovinyldeoxusidine, cytarbine, didanosine, deoxynojirimycin, dideoxycitidine, dideoxyinosine, dideoxynucleoside, desciclovir, deoxyacyclovir, edoxuidine, enviroxime, fiacitabine, foscamet, fialuridine, fluorothymidine, floxuridine, ganciclovir, hypericin, interferon, interleukin, isethionate, idoxuridine, nevirapine, pentamidine, ribavirin, rimantadine, stavirdine, sargramostin, suramin, trichosanthin, trifluorothymidine, tribromothymidine, trichlorothymidine, trisodium phosphomonoformate, vidarabine, zidoviridine, zalcitabine and 3-azido-3-deoxythymidine, and pharmaceutically acceptable salts, analogs, prodrugs or codrugs thereof.

In certain embodiments, the antiviral agent is one which inhibits or reduces HIV infection or susceptibility to HIV infection. Non-nucleoside analogs are preferred and include compounds, such as nevirapine, delavirdine and efavirenz, to name a few. However, nucleoside derivatives, although less preferable, can also be used, including compounds such as 3′azido-3′thymidine (AZT), dideoxyinosine (ddI), 2′,3′-dideoxyadenosine (ddA), 2′,3′-dideoxyguanosine (ddG), 2′,3′-dideoxycytidine (ddC), 2′,3′-dideoxythymidine (ddT), 2′3′-dideoxy-dideoxythymidine (d4T), and 2′-deoxy-3′-thia-cytosine (3TC or lamivudime). Halogenated nucleoside derivatives may also be used including, for example, 2′3′-dideoxy-2′-fluoronucleosides such as 2′,3′-dideoxy-2′-fluoroadenosine, 2′,3′-dideoxy-2′-fluoroinosine, 2′,3′-dideoxy-2′-fluorothymidine, 2′,3′-dideoxy-2′-fluorocytosine, and 2′,3′-dideoxy-2′,3′-didehydro-2′-fluoronucleosides including, but not limited to 2′3′-dideoxy-2′,3′-didehydro-2′-fluorothymidine (Fd4T), 2′3′-dideoxy-2′-beta-fluoroadenosine (F-ddA), 2′3′-dideoxy-2′-beta-fluoro-inosine (F-ddI) and 2′,3′-dideoxy-2′-beta-flurocytosine (F-ddC), and pharmaceutically acceptable salts, analogs, prodrugs or codrugs thereof.

Any pharmaceutically acceptable form of such a compound may be employed in the practice of the present invention, i.e., the free base or a pharmaceutically acceptable salt or ester thereof. Pharmaceutically acceptable salts, for instance, include sulfate, lactate, acetate, stearate, hydrochloride, tartrate, maleate, and the like.

The drug delivery system of the present invention may be administered to a mammalian organism via any route of administration known in the art. Such routes of administration include intraocular, oral, subcutaneous, intramuscular, intraperitoneal, intranasal, dermal, into the brain, including intracranial and intradural, into the joints, including ankles, knees, hips, shoulders, elbows, wrists, directly into tumors, and the like. In addition, one or more of the devices may be administered at one time, or more than one agent may be included in the inner core or reservoir, or more than one reservoir may be provided in a single device.

For systemic relief, the devices may be implanted subcutaneously, intramuscularly, intraarterially, intrathecally, or intraperitoneally. This is the case when devices are to give sustained systemic levels and avoid premature metabolism. In addition, such devices may be administered orally.

For localized drug delivery, the devices may be surgically implanted at or near the desired site of action. This is the case for devices of the present invention used in treating ocular conditions, primary tumors, rheumatic and arthritic conditions, and chronic pain.

The present inventors contemplate a device and method of preparation thereof that is suitable for the controlled and sustained release of an agent or drug effective in obtaining a desired local or systemic physiological or pharmacological effect. In particular, it has been found that by sealing at least one surface of a reservoir of the device with an impermeable member which is capable of supporting its own weight, which has dimensional stability, which has the ability to accept a drug core therein without changing shape, and/or retains its own structural integrity so that the surface area for diffusion does not significantly change, manufacture of the entire device is made simpler and the device is better able to deliver a drug.

The use of a tube of material to hold the drug reservoir during manufacture allows for significantly easier handling of the tube and reservoir, because the tube fully supports both its own weight and the weight of the reservoir. Thus, the tube used in the present invention is not a coating, because a coating cannot support its own weight. Also, this rigid structure allows the use of drug slurries drawn into the tube, which allows the fabrication of longer cylindrical devices. Furthermore, because of the relative ease of manufacturing such devices, more than one reservoir, optionally containing more than one drug, can be incorporated into a single device.

During use of the devices, because the size, shape, or both, of the drug reservoir typically changes as drug diffuses out of the device, the tube which holds the drug reservoir is sufficiently strong or rigid to maintain a diffusion area so that the diffusion rate from the device does not change substantially because of the change in size or surface area of the drug reservoir. By way of example and not of limitation, an exemplary method of ascertaining if the tube is sufficiently rigid is to form a device in accordance with the present invention, and to measure the diffusion rate of the drug from the device over time. If the diffusion rate changes more than 50% from the diffusion rate expected based on the chemical potential gradient across the device at any particular time, the tube has changed shape and is not sufficiently rigid. Another exemplary test is to visually inspect the device as the drug diffuses over time, looking for signs that the tube has collapsed in part or in full.

The use of permeable and impermeable tubes in accordance with the present invention provides flow resistance to reverse flow, i.e., flow back into the device. The tube or tubes assist in preventing large proteins from solubilizing the drug in the drug reservoir. Also, the tube or tubes assist in preventing oxidation and protein lysis, as well as preventing other biological agents from entering the reservoir and eroding the drug therein.

Permeability is necessarily a relative term. As used herein, the term “permeable” is intended to mean permeable or substantially permeable to a substance, which is typically the drug that the device delivers unless otherwise indicated (for example, where a membrane is permeable to a biological fluid from the environment into which a device is delivered). As used herein, the term “impermeable” is intended to mean impermeable or substantially impermeable to a substance, which is typically the drug that the device delivers unless otherwise indicated (for example, where a membrane is impermeable to a biological fluid from the environment into which a device is delivered). The term “semi-permeable” is intended to mean selectively permeable to at least one substance but not others. It will be appreciated that in certain cases, a membrane may be permeable to a drug, and also substantially control a rate at which drug diffuses or otherwise passes through the membrane. Consequently, a permeable membrane may also be a release-rate-limiting or release-rate-controlling membrane, and in certain circumstances, permeability of such a membrane may be one of the most significant characteristics controlling release rate for a device.

Referring to the drawing figures, like reference numerals designate identical or corresponding elements throughout the several figures.

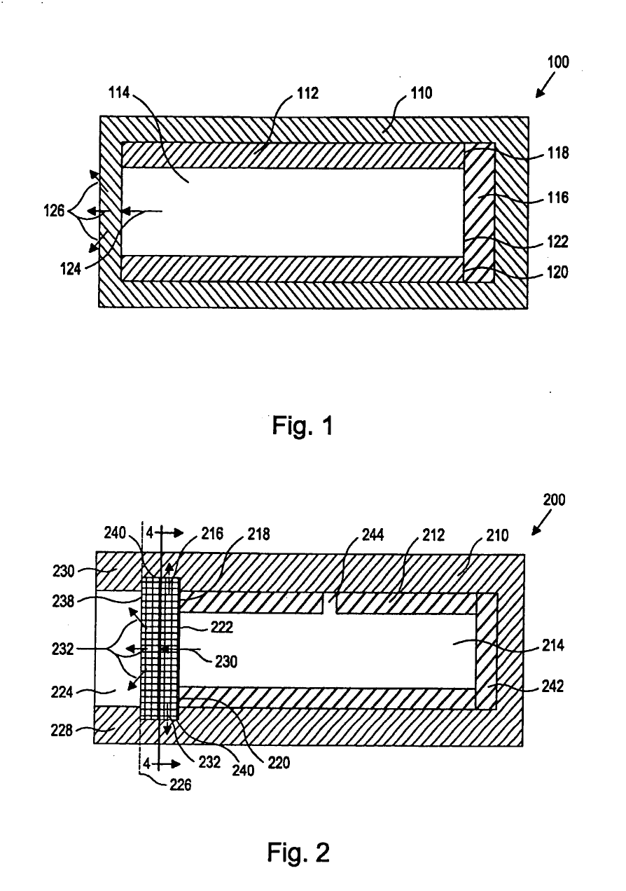

FIG. 1 illustrates a longitudinal cross sectional view of a drug delivery device 100 in accordance with the present invention. Device 100 includes an outer layer 110, an inner tube 112, a reservoir, drug core, drug supply, drug depot, drug matrix, and/or drug in suspension 114, and an inner cap 116. Outer layer 110 is preferably a permeable layer, that is, the outer layer is permeable to the drug contained within reservoir 114. Cap 116 is positioned at one end of tube 112. Cap 116 is preferably formed of an impermeable material, that is, the cap is not permeable to the drug contained within reservoir 114. Cap 116 is joined at end 118, 120 of inner tube 112, so that the cap and the inner tube together close off a space in the tube in which reservoir 114 is positioned, and together the cap and inner tube form a cup- or vessel-shaped member. Inner tube 112 and cap 116 can be formed separately and assembled together, or the inner tube and the cap can be formed as a single, integral, monolithic element.

Outer layer 110 at least partially, and preferably completely, surrounds both tube 112 and cap 116, as illustrated in FIG. 1. While it is sufficient for outer layer 110 to only partially cover tube 112 and cap 116, and in particular the opposite ends of device 100, the outer layer is preferably formed to completely envelop both the tube and cap to provide structural integrity to the device, and to facilitate further manufacturing and handling because the device is less prone to break and fall apart. While FIG. 1 illustrates cap 116 having an outer diameter the same as the outer diameter of inner tube 112, the cap can be sized somewhat smaller or larger than the outer diameter of the inner tube within the spirit and scope of the present invention.

Reservoir 114 is positioned inside inner tube 112, as described above. A first end 122 abuts against cap 116, and is effectively sealed by the cap from diffusing drug therethrough. On the end of reservoir 114 opposite cap 116, the reservoir is preferably in direct contact with outer layer 110. As will be readily appreciated by one of ordinary skill in the art, as drug is released from reservoir 114, the reservoir may shrink or otherwise change shape, and therefore may not fully or directly contact outer layer 110 at the end of the reservoir opposite cap 116. As outer layer 110 is permeable to the drug in reservoir 114, the drug is free to diffuse out of the reservoir along a first flow path 124 into portions of outer layer 110 immediately adjacent to the open end of the reservoir. From outer layer 110, the drug is free to diffuse along flow paths 126 out of the outer layer and into the tissue or other anatomical structure in which device 100 is inserted or implanted. Optionally, holes can be formed through inner layer 112 to add additional flow paths 126 between reservoir 114 and permeable outer layer 110.

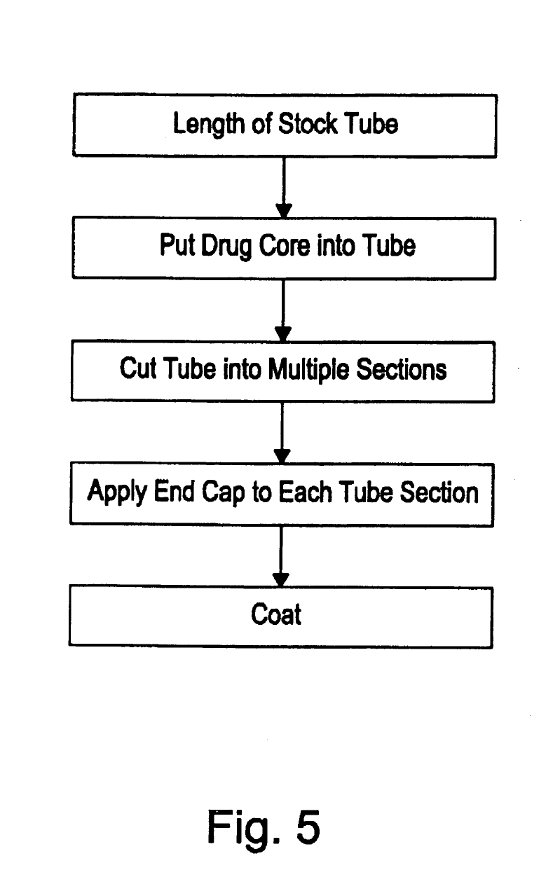

As discussed above, by providing inner tube 112 of a relatively rigid material, it is possible to more easily manufacture device 100. By way of example only and not of limitation, referring to FIG. 5, according to a first embodiment of a process of forming device 100, a length of tube stock material is taken as the starting material. Into the open end of tube 112, opposite cap 116, a drug reservoir 114 is inserted, injected, or otherwise positioned, depending on how viscous the drug reservoir material is when positioned in the tube. If reservoir 114 is relatively stiff, i.e., is very viscous or solid, the reservoir can be inserted into tube 112, as with a plunger, pushrod, or the like. If reservoir 114 is relatively flaccid or fluid, i.e., is not very viscous, the reservoir can be poured, injected, or drawn into the tube (e.g., by vacuum). The length of tube, including the drug core, is then cut into multiple sections, each of which form a tube 112. Cap 116 is joined to one end of tube 112, thus forming a closed, cup- or vessel-like structure. Thereafter, owing to the relative rigidity of inner tube 112, the inner tube and cap 116 can be handled with relative ease, because the inner tube is sized and formed of a material so that it is capable of supporting its own weight, the weight of cap 116, and the weight of reservoir 114, without collapsing. Thereafter, the tube can be coated.

According to yet another embodiment of a process for manufacturing such a device, reservoir 114 can be inserted into a mold, along with cap 116, and inner tube 112 can be molded around the reservoir and cap. Further alternatively, cap 116 can be formed integrally with inner tube 112.

By way of contrast, prior devices, including those which include merely a coating around a drug-containing reservoir, at this stage in the manufacturing process must be specially handled by, for example, forming and placing the reservoir in a carrier which supports the coating and reservoir during handling. As will be readily appreciated by one of ordinary skill in the art, elimination of such additional manufacturing steps and components simplifies the manufacturing process, which in turn can lead to improvements in rejection rates and reductions in costs.

FIG. 1 illustrates only the positions of the several components of device 100 relative to one another, and for ease of illustration shows outer layer 110 and inner tube 112 as having approximately the same wall thickness. While the walls of outer layer 110 and inner tube 112 may be of approximately the same thickness, the inner tube's wall thickness can be significantly thinner or thicker than that of the outer layer within the spirit and scope of the present invention. Additionally, device 100 is preferably cylindrical in shape, for which a transverse cross-section (not illustrated) will show circular cross-sections of the device. While it is preferred to manufacture device 100 as a cylinder with circular cross-sections, it is also within the scope of the present invention to provide cap 116, reservoir 114, inner tube 112, and/or outer layer 110 with other cross-sections, such as ovals, ellipses, rectangles, including squares, triangles, as well as any other regular polygon or irregular shapes. Furthermore, device 100 can optionally further include a second cap (not illustrated) on the end opposite cap 116, such a second cap could be used to facilitate handling of the device during fabrication, and would include at least one through hole for allowing drug from reservoir 114 to flow from the device.

FIG. 2 illustrates a device 200 in accordance with a second exemplary embodiment of the present invention. Device 200 includes an impermeable inner tube 212, a reservoir 214, and a permeable plug 216. Device 200 optionally and preferably includes an impermeable outer layer 210, which adds mechanical integrity and dimensional stability to the device, and aids in manufacturing and handling the device. As illustrated in FIG. 2, reservoir 214 is positioned in the interior of inner tube 212, in a fashion similar to reservoir 114 and inner tube 112, described above. Plug 216 is positioned at one end of inner tube 212, and is joined to the inner tube at end 218, 220 of the inner tube. While plug 216 may extend radially beyond inner tube 212, as illustrated in FIG. 2, the plug may alternatively have substantially the same radial extent as, or a slightly smaller radial extent than, the inner tube, within the spirit and scope of the present invention. As plug 216 is permeable to the agent contained in reservoir 214, the agent is free to diffuse through the plug from the reservoir. Plug 216 therefore must have a radial extent which is at least as large as the radial extent of reservoir 214, SO that the only diffusion pathway 230 out of the reservoir is through the plug. On the end of inner tube 212 opposite plug 216, the inner tube is closed off or sealed only by outer layer 210, as described below. Optionally, an impermeable cap 242, which can take the form of a disc, is positioned at the end of reservoir opposite plug 216. When provided, cap 242 and inner tube 212 can be formed separately and assembled together, or the inner tube and the cap can be formed as a single, integral, monolithic element.

Outer tube or layer 210, when provided, at least partially, and preferably completely surrounds or envelops inner tube 212, reservoir 214, plug 216, and optional cap 242, except for an area immediately adjacent to the plug which defines a port 224. Port 224 is, in preferred embodiments, a hole or blind bore which leads to plug 216 from the exterior of the device. As outer layer 210 is formed of a material which is impermeable to the agent in reservoir 214, the ends of inner tube 212 and reservoir 214 opposite plug 216 are effectively sealed off, and do not include a diffusion pathway for the agent to flow from the reservoir. According to a preferred embodiment, port 224 is formed immediately adjacent to plug 216, on an end 238 of the plug opposite end 222 of reservoir 214. Plug 216 and port 224 therefore include diffusion pathways 230, 232, through the plug and out of device 200, respectively.

While port 224 in the embodiment illustrated in FIG. 2 has a radial extent which is approximately the same as inner tube 212, the port can be sized to be larger or smaller, as will be readily apparent to one of ordinary skill in the art. For example, instead of forming port 224 radially between portions 228, 230 of outer layer 210, these portions 228, 230 can be removed up to line 226, to increase the area of port 224. Port 224 can be further enlarged, as by forming outer layer 210 to extend to cover, and therefore seal, only a portion or none of the radial exterior surface 240 of plug 216, thereby increasing the total surface area of port 224 to include a portion or all of the outer surface area of the plug.

In accordance with yet another embodiment of the present invention, port 224 of device 200 can be formed immediately adjacent to radial external surface 240 of plug 216, in addition to or instead of being formed immediately adjacent to end 238 of the plug. As illustrated in FIG. 4, port 224 can include portions 234, 236, which extend radially away from plug 216. These portions can include large, continuous, circumferential and/or longitudinal portions 236 of plug 216 which are not enveloped by outer layer 210, illustrated in the bottom half of FIG. 4, and/or can include numerous smaller, circumferentially spaced apart portions 234, which are illustrated in the top half of FIG. 4. Advantageously, providing port 224 immediately adjacent to radial external surface 240 of plug 216, as numerous, smaller openings 234 to the plug, allows numerous alternative pathways for the agent to diffuse out of device 200 in the event of a blockage of portions of the port. Larger openings 236, however, benefit from a relative ease in manufacturing, because only a single area of plug 216 need be exposed to form port 224.

According to yet another embodiment of the present invention, plug 216 is formed of an impermeable material and outer layer 210 is formed of a permeable material. A hole or holes are formed, e.g., by drilling, through one or more of inner layer 212, cap 242, and plug 216, which permit drug to be released from reservoir 214 through outer layer 210. According to another embodiment, plug 216 is eliminated as a separate member, and permeable outer layer 210 completely envelopes inner tube 212 and cap 242 (if provided). Thus, the diffusion path ways 230, 232 are through outer layer 210, and no separate port, such as port 224, is necessary. By completely enveloping the other structures with outer layer or tube 210, the system 200 is provided with further dimensional stability. Further optionally, plug 216 can be retained, and outer layer 210 can envelop the plug as well.

According to yet another embodiment of the present invention, inner tube 212 is formed of a permeable material, outer layer 210 is formed of an impermeable material, and cap 242 is formed of either a permeable or an impermeable material. Optionally, cap 242 can be eliminated. As described above, as outer layer 210 is impermeable to the agent in reservoir 214, plug 216, port 224, and optional ports 234, 236, are the only pathways for passage of the agent out of device 200.

In a manner similar to that described above with reference to FIG. 1, the use of a relatively rigid inner tube 212 allows device 200 to be more easily manufactured. According to one embodiment of a process for forming device 200, the combination of plug 216 and inner tube 212 is loaded with reservoir 214, similar to how reservoir 114 is loaded into inner tube 112 and cap 116, described above. Thereafter, if provided, outer layer 210 is formed around plug 216, inner tube 212, reservoir 214, and cap 242 when provided, to form an impermeable outer layer, for reasons discussed above. To form port 224, material is then removed from outer layer 210 to expose a portion of or all of the outer surface of plug 216, as described above. Alternatively, port 224 can be formed simultaneously with the formation of outer layer 210, as by masking the desired area of plug 216.

According to yet another embodiment of a process for manufacturing in accordance with the present invention, reservoir 214 can be inserted into a mold, along with plug 216 and cap 242, and inner tube 112 can be molded around the reservoir, plug, and cap.

The shape of device 200 can be, in a manner similar to that described above with respect to device 100, any of a large number of shapes and geometries. Furthermore, both device 100 and device 200 can include more than one reservoir 114, 214, included in more than one inner tube 112, 212, respectively, which multiple reservoirs can include diverse or the same agent or drug for diffusion out of the device. In device 200, multiple reservoirs 214 can be positioned to abut against only a single plug 216, or each reservoir 214 can have a dedicated plug for that reservoir. Such multiple reservoirs can be enveloped in a single outer layer 110, 210, as will be readily appreciated by one of ordinary skill in the art.

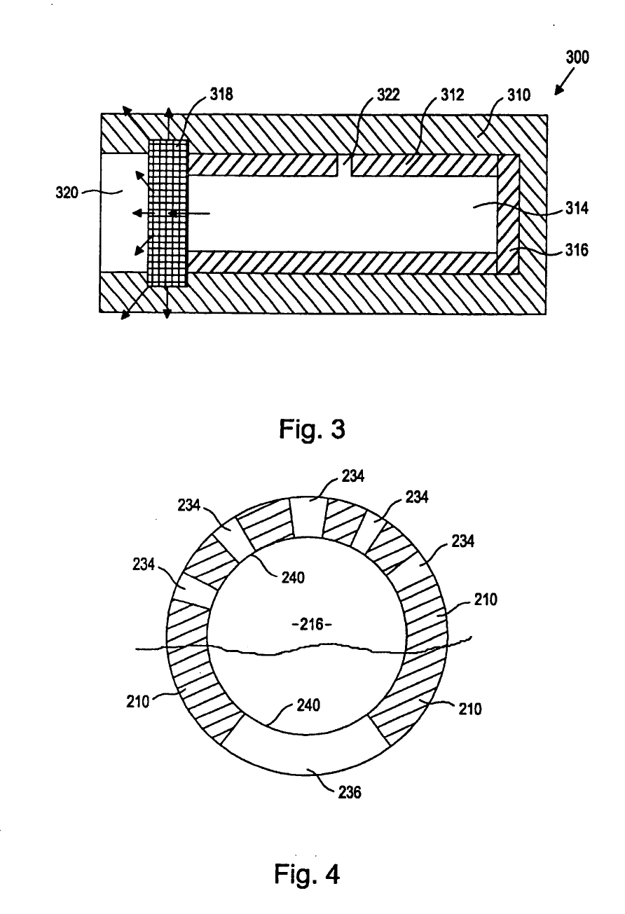

Turning now to FIG. 3, FIG. 3 illustrates a device 300 in accordance with a third exemplary embodiment of the present invention. Device 300 includes a permeable outer layer 310, an impermeable inner tube 312, a reservoir 314, an impermeable cap 316, and a permeable plug 318. A port 320 communicates plug 318 with the exterior of the device, as described above with respect to port 224 and plug 216. Inner tube 312 and cap 316 can be formed separately and assembled together, or the inner tube and the cap can be formed as a single, integral, monolithic element. The provision of permeable outer layer 310 allows the therapeutic agent in reservoir or drug core 314 to flow through the outer layer in addition to port 320, and thus assists in raising the overall delivery rate. Of course, as will be readily appreciated by one of ordinary skill in the art, the permeability of plug 318 is the primary regulator of the drug delivery rate, and is accordingly selected. Additionally, the material out of which outer layer 310 is formed can be specifically chosen for its ability to adhere to the underlying structures, cap 316, tube 312, and plug 318, and to hold the entire structure together. Optionally, a hole or holes 322 can be provided through inner tube 312 to increase the flow rate of drug from reservoir 314.

The invention further relates to a method for treating a mammalian organism to obtain a desired local or systemic physiological or pharmacological effect. The method includes administering the sustained release drug delivery system to the mammalian organism and allowing the agent effective in obtaining the desired local or systemic effect to pass through outer layer 110 of device 100, plug 216 of device 200, or plug 318 and outer layer 310 of device 300 to contact the mammalian organism. The term administering, as used herein, means positioning, inserting, injecting, implanting, or any other means for exposing the device to a mammalian organism. The route of administration depends on a variety of factors including type of response or treatment, type of agent, and preferred site of administration.

The devices in certain embodiments have applicability in providing a controlled and sustained release of agents effective in obtaining a desired local or systemic physiological or pharmacological effect relating at least to the following areas: treatment of cancerous primary tumors, (e.g., glioblastoma), inhibition of neovascularization, including ocular neovascularization, edema, including ocular edema, inflammation, including ocular inflammation, chronic pain, arthritis, rheumatic conditions, hormonal deficiencies such as diabetes and dwarfism, and modification of the immune response such as in the prevention of transplant rejection and in cancer therapy. A wide variety of other disease states may also be prevented or treated using the drug delivery device of the present invention. Such disease states are known by those of ordinary skill in the art. For those not skilled in the art, reference may be made to Goodman and Gilman, The Pharmacological Basis of Therapeutics, 8th Ed., Pergamon Press, N.Y., 1990, and Remington's Pharmaceutical Sciences, 18th Ed., Mack Publishing Co., Easton, Pa., 1990, both of which are incorporated by reference herein.

In addition, the devices are suitable for use in treating mammalian organisms infected with HIV and AIDS-related opportunistic infections such as cytomegalovirus infections, toxoplasmosis, pneumocystis carinii, and mycobacterium avium intercellular.

By “sustained release device” it is meant a device that releases drug over an extended period of time in a controlled fashion. Examples of sustained release devices useful in the present invention may be found in, for example, U.S. Pat. No. 5,378,475, U.S. Pat. No. 5,773,019, and U.S. Pat. No. 5,902,598.

For example, U.S. Pat. No. 5,378,475 (the “'475 patent”) teaches a device includes an inner core or reservoir which contains an agent effective in obtaining a desired effect. The device further includes a first coating layer and a second coating layer. The first coating layer covers only a portion of the inner core and is impermeable to the passage of the agent. The second coating layer covers all of the inner core and the first coating layer and is permeable to the passage of the agent. The portion of the inner core that is not coated with the first coating layer facilitates passage of the agent through the second coating layer.

Specifically, the first coating layer is positioned between the inner core and the second coating layer such that it blocks the passage of the agent through the adjacent portions of the second coating layer thus controlling the rate of passage of the agent.

The first layer must be selected to be impermeable, as described above, to the passage of the agent from the inner core out to adjacent portions of the second coating layer. The purpose is to block the passage of the agent to those portions and thus control the release of the agent out of the drug delivery device.

The composition of the first layer, e.g., the polymer, must be selected so as to allow the above-described controlled release. The preferred composition of the first layer will vary depending on such factors as the active agent, the desired rate of control and the mode of administration. The identity of the active agent is important since the size of the molecule, for instance, is critical in determining the rate of release of the agent into the second layer.

Since the first coating layer is essentially impermeable to the passage of the effective agent, only a portion of the inner core or reservoir may be coated with the first coating layer. Depending on the desired delivery rate of the device the first coating layer may coat only a small portion of the surface area of the inner core for faster release rates of the effective agent or may coat large portions of the surface area of the inner core for slower release rates of the effective agent.

For faster release rates, the first coating layer may coat up to 10% of the surface area of the inner core. Preferably, approximately 5-10% of the surface area of the inner core is coated with the first coating layer for faster release rates.

For slower release rates, the first coating layer may coat at least 10% of the surface area of the inner core. Preferably, at least 25% of the surface area of the inner core is coated with the first coating layer. For even slower release rates, at least 50% of the surface area may be coated. For even slower release rates, at least 75% of the surface area may be coated. For even slower release rates, at least 95% of the surface area may be coated.

Thus, any portion of the surface area of the inner core up to but not including 100% may be coated with the first coating layer as long as the desired rate of release of the agent is obtained.

The first coating may be positioned anywhere on the inner core, including but not limited to the top, bottom or any side of the inner core. In addition, it could be on the top and a side, or the bottom and a side, or the top and the bottom, or on opposite sides or on any combination of the top, bottom or sides.

The second layer of the device of the present invention must be biologically compatible, essentially insoluble in body fluids with which the material will come in contact and permeable to the passage of the agent or composition effective in obtaining the desired effect.

The effective agent diffuses in the direction of lower chemical potential, i.e., toward the exterior surface of the device. At the exterior surface of the device, equilibrium is again established. When the conditions on both sides of the second coating layer are maintained constant, a steady state flux of the effective agent will be established in accordance with Fick's Law of Diffusion. The rate of passage of the drug through the material by diffusion is generally dependent on the solubility of the drug therein, as well as on the thickness of the wall. This means that selection of appropriate materials for fabricating the wall will be dependent on the particular drug to be used.

U.S. Pat. No. 5,773,019 (the “'019 patent”) describes a device including an inner core comprising an effective amount of a low solubility agent, and a non-bioerodible polymer coating layer, the polymer layer permeable to the low solubility agent, wherein the polymer coating layer covers the inner core.

Once implanted, the device gives a continuous supply of the agent to internal regions of the body without requiring additional invasive penetrations into these regions. Instead, the device remains in the body and serves as a continuous source of the agent to the affected area. In another embodiment, the device further comprises a means for attachment, such as an extension of the non-erodible polymer coating layer, a backing member, or a support ring.

The non-bioerodible polymer coating layer may completely or partially cover the inner core. In this regard, any portion of the surface area of the inner core up to and including 100% may be coated with the polymer coating layer as long as the pellet is protected against disintegration, prevented from being physically displaced from its required site, and as long as the polymer coating layer does not adversely retard the release rate.

U.S. Pat. No. 5,902,598 (the “'598 patent”) further teaches a device, in one embodiment, including an inner core or reservoir which contains an agent effective in obtaining the desired effect. The device further includes a first coating layer. The first coating layer is permeable to the passage of the agent. In addition, the device includes a second coating layer which includes at least one impermeable disc and an impermeable polymer. The second coating layer is essentially impermeable to the passage of the agent and covers a portion of the first coating layer and inner core. The second coating layer blocks passage of the agent from the inner core at those sides where it contacts the first coating layer. The remaining portion of the inner core which is not blocked allows a controlled amount of the agent from the inner core to pass into the first coating layer via a passage in the second coating layer, into a third coating layer. The third coating layer is permeable to the passage of the agent and covers essentially the entire second coating layer. The second coating layer is positioned between the inner core and the third coating layer in order to control the rate at which the agent permeates through the third coating layer.

In particular, it has been found that by sealing at least one surface with an impermeable disc, thinner coatings may be utilized. This has the advantage of enabling thinner, shorter devices to be prepared than otherwise possible. A further advantage is that as the material used to prepare the impermeable disc need not be malleable (to facilitate covering of a curved surface); instead relatively hard materials can be used to ease creation of uniform diffusion ports.

The device includes an inner core or reservoir which contains an agent effective in obtaining a desired effect. The device further includes a first coating layer, a second coating layer and a third coating layer. The first coating layer which is permeable to the passage of the effective agent may completely cover the inner core. The second coating layer covers only a portion of the first coating layer and inner core and is impermeable to the passage of the agent. The third coating layer covers all of the first coating layer and second coating layer and is permeable to the passage of the agent. The portion of the first coating layer and inner core that is not coated with the second coating layer facilitates passage of the agent through the third coating layer. Specifically, the second coating layer is positioned between the inner core and the third coating layer such that it blocks the passage of the agent through the adjacent portions of the third coating layer thus controlling the rate of passage of the agent.

Materials that may be suitable for fabricating the device include naturally occurring or synthetic materials that are biologically compatible, and essentially insoluble in body fluids with which the material will come in contact. The use of rapidly dissolving materials or materials highly soluble in fluids are to be avoided since dissolution of the wall would affect the constancy of the drug release, as well as the capability of the system to remain in place for a prolonged period of time. A large number of materials can be used to construct the devices of the present invention. The only requirements are that the materials have the desired inert, non-immunogenic, and permeability characteristics, as described herein.

Materials that may be suitable for fabricating devices 100, 200, and 300 include naturally occurring or synthetic materials that are biologically compatible with body fluids and essentially insoluble in body fluids with which the material will come in contact. The use of rapidly dissolving materials or materials highly soluble in fluids are to be avoided since dissolution of the outer layers 110, 210, 310 would affect the constancy of the drug release, as well as the capability of the system to remain in place for a prolonged period of time.

Specifically, outer layer 210 of device 200 may be made of any of the above listed polymers or any other polymer which is biologically compatible with body fluids and eye tissues, essentially insoluble in body fluids with which the material will come in contact, and essentially impermeable to the passage of the effective agent. The term impermeable, as used herein, means that the layer will not allow passage of the effective agent at a rate required to obtain the desired local or systemic physiological or pharmacological effect.

When inner tube 112, 212, 312 is be selected to be impermeable, as described above, to the passage of the agent from the inner core or reservoir out to adjacent portions of the device, the purpose is to block the passage of the agent to those portions of the device, and thus control the release of the agent out of the drug delivery device through outer layer 110, plug 216, and plug 318.

The composition of outer layer 110, e.g., the polymer, must be selected so as to allow the above-described controlled release. The preferred composition of outer layer 110 and plug 216 will vary depending on such factors as the active agent, the desired rate of control, and the mode of administration. The identity of the active agent is important since the size of the molecule, for instance, is critical in determining the rate of release of the agent into the outer layer 110 and plug 216.

Caps 116, 242, 316 are essentially impermeable to the passage of the effective agent and may cover a portion of the inner tube not covered by the outer layer. The physical properties of the material, preferably a polymer, used for the caps can be selected based on their ability to withstand subsequent processing steps (such as heat curing) without suffering deformation of the device. The material, e.g., polymer, for impermeable outer layer 210 can be selected based on the ease of coating inner tube 212. Cap 116 can be formed of one of a number of materials, including PTFE, polycarbonate, polymethyl methacrylate, polyethylene alcohol, high grades of ethylene vinyl acetate (9% vinyl, content), and polyvinyl alcohol (PVA). Inner tubes 112, 212, 312 can be formed of one of a number of materials, including PTFE, polycarbonate, polymethyl methacrylate, polyethylene alcohol, high grades of ethylene vinyl acetate (9% vinyl, content), and polyvinyl alcohol. Plugs 216, 318 can be formed of one of a number of materials, including cross-linked PVA, as described below.

Outer layers 110, 210, 310, and plugs 216, 318 of the device of the present invention must be biologically compatible with body fluids and tissues, essentially insoluble in body fluids which the material will come in contact, and outer layer 110 and plugs 216, 318 must be permeable to the passage of the agent or composition effective in obtaining the desired effect.

Naturally occurring or synthetic materials that are biologically compatible and essentially insoluble in body fluids which the material will come in contact include, but are not limited to, ethyl vinyl acetate, polyvinyl acetate, cross-linked polyvinyl alcohol, cross-linked polyvinyl butyrate, ethylene ethylacrylate copolymer, polyethyl hexylacrylate, polyvinyl chloride, polyvinyl acetals, plasiticized ethylene vinylacetate copolymer, polyvinyl alcohol, polyvinyl acetate, ethylene vinylchloride copolymer, polyvinyl esters, polyvinylbutyrate, polyvinylformal, polyamides, polymethylmethacrylate, polybutylmethacrylate, plasticized polyvinyl chloride, plasticized nylon, plasticized soft nylon, plasticized polyethylene terephthalate, natural rubber, polyisoprene, polyisobutylene, polybutadiene, polyethylene, polytetrafluoroethylene, polyvinylidene chloride, polyacrylonitrile, cross-linked polyvinylpyrrolidone, polytrifluorochloroethylene, chlorinated polyethylene, poly(1,4′-isopropylidene diphenylene carbonate), vinylidene chloride, acrylonitrile copolymer, vinyl chloride-diethyl fumerale copolymer, silicone rubbers, especially the medical grade polydimethylsiloxanes, ethylene-propylene rubber, silicone-carbonate copolymers, vinylidene chloride-vinyl chloride copolymer, vinyl chloride-acrylonitrile copolymer, vinylidene chloride-acrylonitride copolymer, gold, platinum, and (surgical) stainless steel.

Specifically, the second layer of the device of the present invention may be made of any of the above-listed polymers or any other polymer which is biologically compatible, essentially insoluble in body fluids which the material will come in contact and essentially impermeable to the passage of the effective agent. The term impermeable, as used herein, means that the layer will not allow passage of the effective agent at a rate required to obtain the desired local or systemic physiological or pharmacological effect.

The second layer must be selected to be impermeable, as described above, to the passage of the agent from the inner core out to adjacent portions of the second coating layer. The purpose is to block the passage of the agent to those portions and thus control the release of the agent out of the drug delivery device.

The composition of the second layer, e.g., the polymer, must be selected so as to allow the above-described controlled release. The preferred composition of the second layer will vary depending on such factors as the active agent, the desired rate of control and the mode of administration. The identity of the active agent is important since the size of the molecule, for instance, is critical in determining the rate of release of the agent into the second layer.

Since the second coating layer is essentially impermeable to the passage of the effective agent, only a portion of the inner core or reservoir and first coating layer may be coated with the second coating layer. Depending on the desired delivery rate of the device, the second coating layer may coat only a small portion of the surface area of the inner core for faster release rates of the effective agent or may coat large portions of the surface area of the inner core for slower release rates of the effective agent.

At least 50% of the surface area may be coated by the second coating layer. For slower release rates, at least 75% of the surface area may be coated. For even slower release rates, at least 95% of the surface area may be coated.

Thus, any portion of the surface area of the first coating layer and inner core up to but not including 100% may be coated with the second coating layer as long as the desired rate of release of the agent is obtained.

The second coating, including the impermeable film and impermeable disc, may be positioned anywhere over the inner core and first coating layer, including but not limited to the top, bottom or any side of the first coating layer and inner core. In addition, it could be on the top and a side, or the bottom and a side, or the top and the bottom, or on opposite sides or on any combination of the top, bottom or sides.

The first and third layer of the device of the present invention must be biologically compatible, essentially insoluble in body fluids which the material will come in contact and permeable to the passage of the agent or composition effective in obtaining the desired effect.

The effective agent diffuses in the direction of lower chemical potential, i.e., toward the exterior surface of the device. At the exterior surface of the device, equilibrium is again established. When the conditions on both sides of the third coating layer are maintained constant, a steady state flux of the effective agent will be established in accordance with Fick's Law of Diffusion. The rate of passage of the drug through the material by diffusion is generally dependent on the solubility of the drug therein, as well as on the thickness of the wall. This means that selection of appropriate materials for fabricating the wall will be dependent on the particular drug to be used.

The rate of diffusion of the effective agent through a polymeric layer of the present invention may be determined via diffusion cell studies carried out under sink conditions. In diffusion cell studies carried out under sink conditions, the concentration of drug in the receptor compartment is essentially zero when compared to the high concentration in the donor compartment. Under these conditions, the rate of drug release is given by:

Q/t=(D·K·A·DC)/h

where Q is the amount of drug released, t is time, D is the diffusion coefficient, K is the partition coefficient, A is the surface area, DC is the difference in concentration of the drug across the membrane, and h is the thickness of the membrane.

In the case where the agent diffuses through the layer via water filled pores, there is no partitioning phenomena. Thus, K can be eliminated from the equation. Under sink conditions, if release from the donor side is very slow, the value DC is essentially constant and equal to the concentration of the donor compartment. Release rate therefore becomes dependent on the surface area (A), thickness (h) and diffusivity (D) of the membrane. In the construction of the device of the present invention, the size (and therefore, surface area) is mainly dependent on the size of the effective agent.

Thus, permeability values may be obtained from the slopes of a Q versus time plot. The permeability P, can be related to the diffusion coefficient D, by:

P=(K·D)/h

Once the permeability is established for the coating permeable to the passage of the agent, the surface area of the agent that must be coated with the coating impermeable to the passage of the agent may be determined. This is done by progressively reducing the available surface area until the desired release rate is obtained.

Exemplary microporous materials suitable for use as a first and third coating layer, for instance, are described in U.S. Pat. No. 4,014,335 which is incorporated herein by reference in its entirety. These materials include cross-linked polyvinyl alcohol, polyolefins or polyvinyl chlorides or cross-linked gelatins, regenerated, insoluble, nonerodible cellulose, acylated cellulose, esterified celluloses, cellulose acetate propionate, cellulose acetate butyrate, cellulose acetate phthalate, cellulose acetate diethyl-aminoacetate, polyurethanes, polycarbonates, and microporous polymers formed by co-precipitation of a polycation and a polyanion modified insoluble collagen. Cross-linked polyvinyl alcohol is preferred. The third coating layer is selected so as to slow release of the agent from the inner core into contact with a mammalian organism, e.g., a human. The third coating layer need not provide gradual release or control of the agent into the biological environment, however, the third coating layer may be advantageously selected to also have that property or feature.

The devices of the invention may be made in a wide variety of ways, such as by obtaining an effective amount of the agent and compressing the agent to a desired shape. Once shaped, the first coating layer may be applied. The first coating layer may be applied by dipping the device one or more times in a solution containing the desired polymer. Optionally, the first coating may be applied by dropping, spraying, brushing or other means of coating the outer surface of the device with the polymer solution. When using a polyvinyl alcohol solution to obtain the second coating layer, the desired thickness may be obtained by applying several coats. Each coat may be dried prior to applying the next coat. Finally, the device may be heated to adjust the permeability of the outer coating.

The impermeable disc may be applied directly over the first layer before coating with the impermeable polymer layer. In the case of a cylindrical core, an impermeable film may be wrapped around the core after discs are applied to one or both ends. Thus, the second coating layer includes both the impermeable film and the impermeable discs. By sealing at least one surface with an impermeable disc, thinner layers may be utilized. This has the advantage of enabling thinner, shorter devices to be prepared than otherwise possible.

Impermeable polymer layers in devices in accordance with the present invention should be thick enough to prevent release of drug across them except for the area not covered (the diffusion layer or port), e.g., port 224. Due to the desirability of minimizing the size of the implantable devices, the thickness of an impermeable layer therefore can be between about 0.01 and about 2 millimeters, preferably between about 0.01 and about 0.5 millimeters, most preferably between about 0.01 and about 0.2 millimeters.

The impermeable disc (e.g., caps 116, 242) should also be thick enough to prevent drug release across it save through a specifically prepared membrane or port. Due to the desirability of minimizing the size of the implants, the thickness of the impermeable disc can be 0.01 to 2 millimeters, preferably between about 0.01 and about 0.5 millimeters, most preferably between about 0.01 and about 0.2 millimeters.

Once the second coating layer, including the impermeable disc(s), is applied to the device, the third coating layer may be applied. The third coating may be applied by dipping the device one or more times in a solution containing the desired polymer. Optionally, the third coating layer may be applied by dropping, spraying, brushing or other means of coating the outer surface of the device with the polymer solution. When using a polyvinyl alcohol solution to obtain the third coating layer, the desired thickness may be obtained by applying several coats. Each coat may be dried prior to applying the next coat. Finally, the device may be heated to adjust the permeability of the outer coating.

In still other embodiments, the sustained release device can be formed by co-extrusion of a drug-containing inner core and a self-supportable outer skin. The device is preferably tube-shaped although products with other cross sections can be prepared. Such devices and methods for manufacturing such device are described in U.S. application Ser. No. 10/428,214 (“the '214 application”), filed May 2, 2003, and U.S. Application entitled “Injectable Sustained Release Drug Delivery Devices,” (Chou et al.), filed Nov. 13, 2003 (“the Nov. 13, 2003 application”), both of which are incorporated by reference in its entirety herein. Drug delivery devices, including injectable drug delivery devices, of the present invention that are formed in accordance with the methods described in the '214 application and Nov. 13, 2003 Application include a core containing one or more antiviral dugs and one or more polymers. The core may be surrounded by one or more polymer outer layers. In certain embodiments, the device is formed by extruding or otherwise preforming a polymeric skin for a drug core. The drug core may be co-extruded with the skin, or inserted into the skin after the skin has been extruded, and possibly cured. In other embodiments, the drug core may be coated with one or more polymer coatings. These techniques may be usefully applied to fabricate devices having a wide array of drug formulations and skins that can be selected to control the release rate profile and various other properties of the drugs in the drug core in a form suitable for injection using standard or non-standard gauge needles. The device may be formed by combining at least one polymer, at least one drug, and at least one liquid solvent to form a liquid suspension or solution wherein, upon injection, such suspension or solution under goes a phase change and forms a gel. The configuration may provide for controlled release of the drug(s) for an extended period.

In embodiments using a skin, the skin may be permeable, semi-permeable, or impermeable to the drug, or to the fluid environment to which the device may be exposed. The drug core may include a polymer matrix that does not significantly affect the release rate of the drug. Alternatively, such a polymer matrix may affect the release rate of the drug. The skin, the polymer matrix of the drug core, or both may be bioerodible. The device may be fabricated as an extended mass that is segmented into drug delivery devices, which may be left uncoated so that the drug core is exposed on all sides or (where a skin is used) at the ends of each segment, or coated with a layer such as a layer that is permeable to the drug, semi-permeable to the drug, impermeable, or bioerodible.

In other embodiments, the drug-containing core may comprise a biocompatible fluid or oil combined with a biocompatible solid (e.g., a bioerodible polymer) and an antiviral agent. In certain embodiments, the inner core may be delivered as a gel while, in certain other embodiments, the inner core may be delivered as a particulate or a liquid that converts to a gel upon contact with water or physiological fluid. Examples of this type of system are described for example, in U.S. Provisional Application No. 60/501,947 (“the '947 application”), filed Sep. 11, 2003. The '947 application also provides for the delivery of injectable liquids that, upon injection, undergo a phase transition and are transformed in situ into gel delivery vehicles. Such liquids may be employed with the injectable devices described herein.

Injectable in situ gelling compositions may be used with the systems described herein, comprising an antiviral agent, a biocompatible solvent (e.g., a polyethylene glycol (PEG)), and a biocompatible and bioerodible polymer. Certain embodiments of this formulation may be particularly suitable, such as those that provide for the injection of solid drug particles that are dissolved, dispersed, or suspended in the PEG, and embodiments that allow for the injection of a polymeric drug-containing gel into a patient. Examples of injectable in situ gelling compositions may be found in U.S. Provisional App. No. 60/482,677, filed Jun. 26, 2003.

The above description of how to make the devices of the present invention is merely illustrative and should not be considered as limiting the scope of the invention in any way, as various compositions are well known by those skilled in the art. In particular, the methods of making the device depends on the identity of the active agent and polymers selected. Given the active agent, the composition of the outer layers, the inner tube, the plug, and the cap, one skilled in the art could easily make the devices of the present invention using conventional coating techniques.

The phrase “pharmaceutically acceptable carrier” as used herein means a pharmaceutically acceptable material, composition or vehicle, such as a liquid or solid filler, diluent, excipient, or encapsulating material, involved in carrying or transporting the subject antagonists from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; and (16) other non-toxic compatible substances employed in pharmaceutical formulations.

Experiments

### EXAMPLE 1

Test Implant Characterization/Stability

| | |
|---|---|
| Test Device: | Test Implants |
| | 20 mg Nevirapine [NVP] Implants |
| | Each implant contained 20 mg nevirapine, 1.0 mg |
| | PEG 3350, and 0.4 mg of magnesium stearate. The |
| | implant was dip-coated in PVA before being |
| | inserted into a precut silicone tube. The |
| | approximate size of each implant was 5 mm |
| | (length) × 2 mm (diameter). |
| Control Device: | Sham Implants |
| | Silicone tubing identical to that used to house the |
| | Test Implants |

Summary

The primary objective of this study was to evaluate plasma levels of nevirapine (a non-nucleoside reverse transcriptase inhibitor with activity against Human Immunodeficiency Virus Type 1[HIV-1]) in female Sprague-Dawley rats, following subcutaneous implantation of a test implant. Each implant contained 20 mg of nevirapine, 1.0 mg PEG 3350, and 0.4 mg of magnesium stearate and was dip-coated in PVA before being inserted into a precut silicone tube. Sham implants consisted of silicone tubing identical to that used to house the test implants. Five female Sprague-Dawley rats were implanted with six sham implants each, and fifteen rats were implanted with six test implants. Toxicity was assessed through evaluation of clinical observations, body weights, and macroscopic pathology of the implant site, and blood samples were collected for analysis of plasma nevirapine concentrations.

The mean body weight of all groups increased over the period of the study. There was no difference between the mean body weights of the sham- or the test-implanted animals.

The peak-mean plasma concentration of nevirapine was 413±138 ng/mL at 7 hours post implantation. The plasma levels declined over the remainder of the study. The plasma levels apparently reached steady state between Day 70 and Day 91 of the study (61.5±6.1 and 61.5±17.6 ng/mL, respectively). The decrease in plasma nevirapine over time may also have resulted from repositioning/migration of the test implants. At the time of necropsy, the test implants had assumed new configurations relative to each other. The repositioning may have physically impaired the release of nevirapine from the ends of the implants.

The test implant was not associated with any abnormal clinical observations, body weights, or macroscopic lesions.

Introduction

The objective of this study was to determine the plasma concentrations of nevirapine following subcutaneous implantation of a nevirapine-containing test implant in rats.

Experimental Design

Overview

The study consisted of one group of five and one group of fifteen female Sprague-Dawley rats; Groups 1 and 2, respectively. Group 2 animals were surgically implanted with the test device (nevirapine implant; 20 mg), and Group 1 received the control device (sham implant). Each animal received six subcutaneous implants, which were placed adjacent to each other in the inter-scapular region. For Group 2 animals, each test implant was composed of 20 mg nevirapine, 1.0 mg PEG 3350, and 0.4 mg of magnesium stearate. The implant was dip-coated in PVA and inserted into a precut silicone tube designed to release 100 ng of nevirapine per day. The total anticipated dose level for Group 2 animals was 600 ng nevirapine/day. Group 1 animals received sham implants composed of silicone tubing identical to that used to house the test implants. The day of surgical implantation was designated Day 1. At protocol-specified time points, clinical observations were performed and body weights were recorded. Blood samples were collected for analysis of plasma nevirapine concentrations. All animals were euthanized on Day 91 and a limited necropsy and tissue collection were performed.

Study Design

Text TABLE 1
Study Design

| | | | Total | Daily Dose | | |
|---|---|---|---|---|---|---|
| Group | Number of | | Implanted | Level* | Dosing | Necropsy |
| Number | Females | Test Device | Dose (mg) | (ng/day) | Regimen | Day |
| 1 | 5 | Sham Implant | 0 | 0 | Subcutaneous | 91 |
| 2 | 15 | Test Implant | 120 | 600 | implantation | |
| | | | | | on Day 1 | |

*Each test implant was designed to release 100 ng/day of nevirapine. A total of six implants were placed into each rat for a total anticipated release of 600 ng nevirapine/day. A total of six sham implants were placed into each Group 1 rat.

Materials and Methods

Device Implantation: Preoperative Procedures

Analgesia, Anesthesia, and Antibiotic Therapy

The animals were pre-anesthetized with atropine sulfate (0.4 mg/kg, subcutaneously, [SC]). Approximately 10-30 minutes later, the animals were anesthetized with a combination of ketamine/medetomidine (60 mg/kg and 0.3 mg/kg, respectively, intramuscularly, [IM]). Drugs for appropriate anesthetic management were available for administration if indicated. The drug, dose, route, and site of administration were documented in the surgical records.

Surgical Preparation

An ophthalmic ointment was administered to each eye. The fur was removed from the inter-scapular region, extending laterally on both sides to the lateral midline. Any excess fur was brushed or vacuumed off. The animal was placed in ventral recumbency on a circulating hot water pad in order to help maintain body heat. The surgical area was then gently wiped with 70% isopropyl alcohol which was allowed to dry. DuraPrep™, or similar solution, was then applied to the area and also allowed to dry.

Blood Sample Collection

Blood samples were collected according to the schedule in Text Table 2. Blood volumes represent whole blood and are approximate amounts. Samples were collected by puncture of the retro-orbital sinus/plexus after the animals had been anesthetized with carbon dioxide (CO₂). All animals were bled to apply the same stress from anesthesia and blood loss, however blood was analyzed for Group 2 only with the exception of the 7-hour samples from Animal Nos. 4 and 5, which were also processed and analyzed. Following collection, Group 2 samples were transferred to the appropriate laboratory for processing and analysis.

TEXT TABLE 2

| | Number of | BAC |
|---|---|---|
| Time Point | Animals | Toxicokinetics |
| Day 1 at 1, 3, 7, 12, and 28^{a} hours post | 20^{b,c} | X |
| implantation | | |
| Days 3, 7, 14, 28, 42, 56, 70, 84, and 91 | 20 | X |
| Blood Sample Collection Schedule | | |

| | |
|---|---|
| Volume of Whole Blood/ | 0.75 mL/ |
| Time Point | animal |
| Anticoagulant | EDTA |

^{a}The 28-hour collection was originally scheduled for 24 hours post dosing.
^{b}The first 20 implanted rats (5 sham implant and 15 test implant) from which 1-hour blood samples were collected were placed on study.
^{c}All 20 rats were bled at the 1-hour time point. Seven of the Group 2 rats and three of the Group 1 rats were bled at 3 and 12 hours. The other eight Group 2 rats and two Group 1 rats ere bled at the 7- and 28-hour time points.

Bioanalytical Chemistry

The blood samples from all Group 2 animals and the 7-hour samples for Group I Animal Nos. 4 and 5 were centrifuged, and plasma was collected and placed in a ≦−70° C. freezer until analysis by the Test Facility's Bioanalytical Chemistry Department. Plasma samples were analyzed for nevirapine concentrations using a method validated by the Test Facility.

Euthanasia

All animals were euthanized on Day 91 via carbon dioxide asphyxiation followed by thorocotomy. All euthanasia procedures were conducted in accordance with accepted American Veterinary Medical Association (AVMA) guidelines.

Necropsy

A limited necropsy, defined as an examination of the external surface of the body, the implant site, underlying muscle, and surrounding tissue, was performed on all animals. The implants were retrieved, gently cleaned of adhering tissue, and stored dry and frozen at ≦−20° C. pending shipment to the Sterigenics (Charlotte, N.C.).

The implant site (with underlying muscle layers), including the total diameter encompassed by the implants plus a few millimeters of surrounding tissue, was examined in situ, dissected free, and fixed in 10% neutral buffered formalin or other suitable fixative for possible histopathological examination. Observations noted at necropsy were recorded.

Statistical Analysis

Quantitative analysis of body weights consisted of the comparison of the treated group with controls at corresponding time points. To determine the appropriate statistical test, each data set was subjected to a statistical decision tree developed by the Test Facility using SAS®, a software system for data analysis. First, the distribution of each data set was assessed for homogeneity of variance using the Bartlett Test. If this test indicated homogeneity of variance (p>0.05) then a parametric distribution was assumed and a one-way analysis of variance (ANOVA) was performed.

A 95% confidence level (p<0.05) was the criterion for statistical significance in all quantitative tests performed in this study. Statistical significance is indicated in the tables and appendices of this report using a dagger (t) adjacent to the mean value. Tables and appendices present group means and standard deviations.

Results

Surgical Implantation

The test or control implants were surgically implanted successfully in all animals.

Clinical Observations

Individual clinical observations are summarized in Table 1.

Clinical observations were normal for all animals in Group 1 for the duration of the study. In Group 2, there were observations of lacrimation, dry red material, and opaque or protruding eyes. These observations always occurred in the right eye and were linked to blood collection. Animal No. 13 had a damaged/abnormal incisor and exhibited skin swelling around the mouth on Days 16 and 23, and was noted to be thin on Day 16 and Day 23. This animal was given moistened food for the remainder of the study. This animal also had a rough hair coat on Days 79, 86, and 91.

Body Weights

Group means body weights are summarized in Table 2.

The mean body weight of animals in Group 1 and Group 2 increased over the duration of the study. There were no differences in mean body weight in Group 2 as compared to the mean body weights of Group 1 at any time points in the study. The nevirapine implants did not affect body weight.

Plasma Nevirapine Concentrations

The HPLC/MS/MS method used to analyze the plasma levels of nevirapine was validated over a range of 20.0 ng/mL to 5000 ng/mL, and samples with levels less than 20 mg/mL were classified as below the limit of quantification (BQL).

Blood samples for Group 1 Animal Nos. 4 and 5, taken at 7 hours post implantation were also processed and analyzed. The levels of nevirapine found in Animal Nos. 4 and 5 were BQL. The 24-hour post-dosing blood sample was collected 28 hours post dosing.

For the purposes of determining the group mean and standard deviation, BQL was set to zero. After surgical implantation of the nevirapine-containing test implants, the blood levels of nevirapine increased steadily. At one hour post implantation, ten of the fifteen animals had BQL plasma levels, and the mean was 9.9±15.0 ng/ml. The peak mean plasma concentration of nevirapine of 413±138 ng/mL at 7 hours post implantation. The plasma levels declined over the remainder of the study. The plasma levels were 297±77.0, 176±40.8, and 123±25.7 ng/mL at 12 and 28 hours, and on Day 3, respectively. The blood levels decreased between Day 7 and Day 91. The plasma levels apparently reached steady state between Day 70 and Day 91 of the study (61.5±6.1 and 61.5±17.6 ng/mL, respectively).

Another possible explanation for the decrease in plasma nevirapine levels over time is repositioning of the test implants. At the time of necropsy, the test implants had assumed new configurations relative to each other. This repositioning may have physically impaired release of nevirapine from the ends of some of the implants.

TEXT TABLE 3
Chronological Plasma Nevirapine Concentrations (ng/mL)

| | Time | | | | | | | | | | | | | | | | | |
|---|---|---|---|---|---|---|---|---|---|---|---|---|---|---|---|---|---|---|
| | after | | | | | | | | | | | | | | | | | |
| | Dosing | Animal Number^{a} | | | | | | | | | | | | | | | | |
| Day | (hr) | 6 | 7 | 8 | 9 | 10 | 11 | 12 | 13 | 14 | 15 | 16 | 17 | 18 | 19 | 20 | Mean^{b} | SD^{b} |
| 1 | 1 | 28.1 | BQL | 20 | 27.9 | BQL | BQL | BQL | 40.1 | BQL | BQL | BQL | BQL | BQL | BQL | 32.5 | 9.9 | 15.0 |
| | 3 | 184 | 88 | 158 | 174 | 119 | 73.1 | 189 | N/A | N/A | N/A | N/A | N/A | N/A | N/A | N/A | 141 | 47.3 |
| | 7 | N/A | N/A | N/A | N/A | N/A | N/A | N/A | 557 | 622 | 473 | 341 | 458 | 307 | 211 | 337 | 413 | 138 |
| | 12 | 412 | 262 | 370 | 224 | 332 | 203 | 277 | N/A | N/A | N/A | N/A | N/A | N/A | N/A | N/A | 297 | 77.0 |
| 2 | 28 | N/A | N/A | N/A | N/A | N/A | N/A | N/A | 196 | 198 | 234 | 197 | 156 | 95.6 | 166 | 172 | 176 | 40.8 |
| 3 | | 109 | 120 | 174 | 97.4 | 146 | 98.4 | 82.6 | 137 | 131 | 160 | 138 | 119 | 94.3 | 110 | 135 | 123 | 25.7 |
| 7 | | 74.2 | 130 | BQL | 89.5 | 80.7 | 99.1 | 156 | 112 | 192 | 107 | 144 | 74.9 | 116 | 144 | 114 | 109 | 44.4 |
| 14 | | 91.6 | 158 | 89 | 71.1 | 124 | 120 | 98.9 | 133 | 136 | 115 | 126 | 66.8 | 91.7 | 112 | 101 | 109 | 25.0 |
| 28 | | 106 | 89.1 | 93.9 | 78.4 | 94.9 | 77.5 | 76 | 84.8 | 115 | 101 | 81.8 | 70.7 | 77.4 | 86.3 | 93.1 | 88.4 | 12.4 |
| 42 | | 77.2 | 89.3 | 80 | 51.6 | 82.9 | 67.6 | 64.4 | 73.7 | 102 | 89.5 | 77.4 | 61.9 | 60 | 93.2 | 86.8 | 77.2 | 14.0 |
| 56 | | 94.6 | 71.1 | 91.1 | 58.9 | 88.9 | 69.1 | 66.2 | 70.8 | 93 | 78.3 | 71.9 | 64.5 | 76.9 | 79.1 | 93.2 | 77.8 | 11.7 |
| 70 | | 68.3 | 67.2 | 67.2 | 53.1 | 61.9 | 59.4 | 55.8 | 53.8 | 69.1 | 67.7 | 54.5 | 53.3 | 61 | 65.2 | 65.6 | 61.5 | 6.1 |
| 84 | | 61.1 | 61.7 | 65.6 | 41.3 | 50.4 | 48.2 | 55.2 | 48 | 68.3 | 49.5 | 57.3 | 47.9 | 48.4 | 56.1 | 53.3 | 54.2 | 7.6 |
| 91 | | 63.3 | 65.3 | 67.3 | 56.1 | 45.8 | 53.9 | 65.3 | 45.9 | 69.9 | 117 | 44.1 | 50.4 | 54.2 | 56.7 | 67.1 | 61.5 | 17.6 |

BQL = Below quantitation limit;
N/A = Not applicable
^{a}All animals in Group 1 (Animals 1-5 were BQL at all timepoints tested).
^{b}For the purpose of determining the mean and standard deviation, BQL was set to zero.

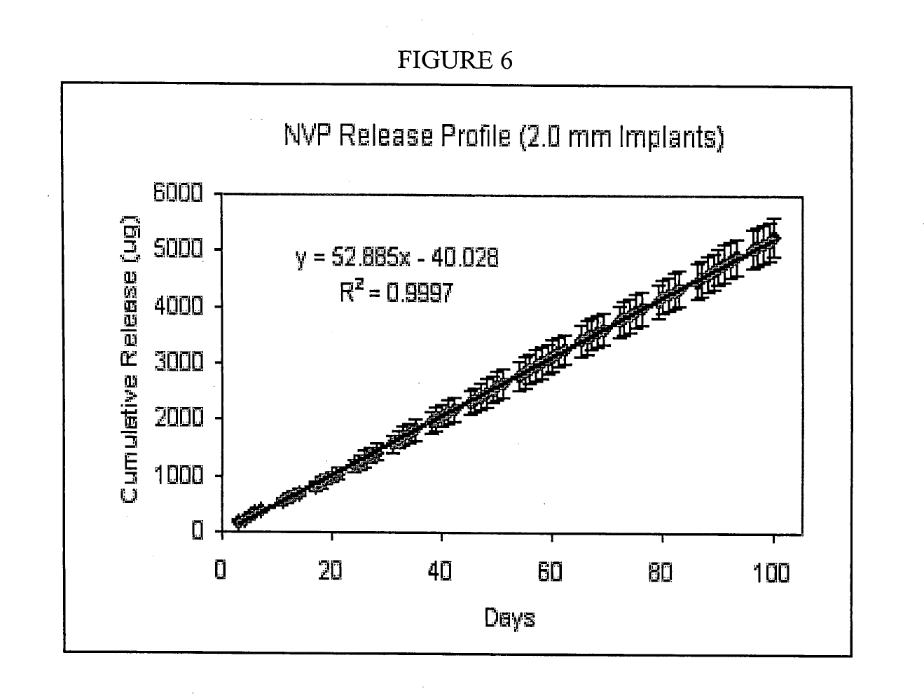

FIG. 6 shows the in vitro release profile of the 2.0 mm NVP implant in 0.1M phosphate buffer (pH 7.4) at 37° C.

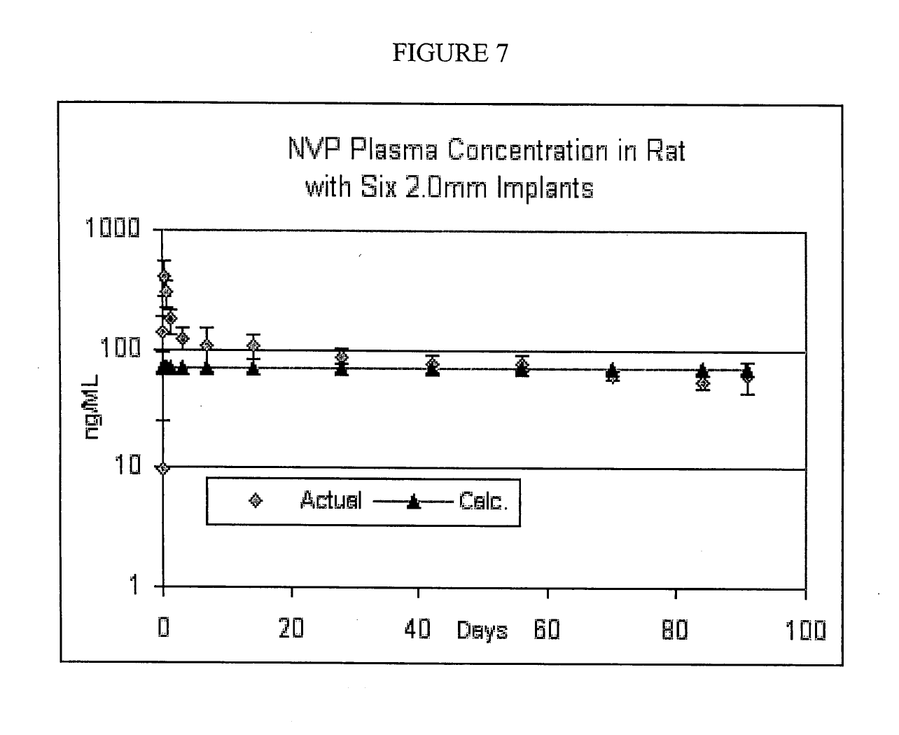

FIG. 7 shows the NVP plasma concentration, from table above, in rats (line marked with diamonds) with six 2.0 mm implants surgically inserted subcutaneously, in comparison with the calculated NVP plasma level (line marked with triangles).

Calculation of Nevirapine (NVP) Plasma Concentration in Rats:

Based on the in vitro release rate (kr), animal body weight (W, 300 gm) and known NVP PK-data [apparent volume of distribution (Vₛₛ): 984 ml/kg and elimination constant (kₑₗ): 0.629 hr⁻¹ in rats] and assuming that the PK follows a one-compartment model, the NVP plasma concentration (C) in rats at steady state can be calculated using the following equation:

C=kᵣ/(kₑₗWVₛₛ)

With an in vitro release rate of 52.9 ug/day (see FIG. 6) for the NVP implant, at a steady state, a NVP plasma concentration of 71 ng/ml is expected for rats with six 2.0 mm implant having release ports on the shell. The calculated NVP concentrations are displayed in FIG. 7 (line marked with triangle).

Conclusion

The primary objective of this study was to evaluate plasma levels of nevirapine (a non-nucleoside reverse transcriptase inhibitor with activity against Human Immunodeficiency Virus Type 1 [HIV-1]) in female Sprague-Dawley rats, following subcutaneous implantation of a nevirapine-containing implant. Each implant contained 20 mg of nevirapine, 1.0 mg PEG 3350, and 0.4 mg of magnesium stearate and was dip-coated in PVA before being inserted into a precut silicone tube. Sham implants consisted of silicone tubing identical to that used to house the test implants. Five female Sprague-Dawley rats were implanted with six sham implants each, and fifteen rats were implanted with six test implants. Toxicity was assessed through evaluation of clinical observations, body weights, and macroscopic pathology of the implant site, and blood samples were collected for analysis of plasma nevirapine concentrations.

The peak mean plasma concentration of nevirapine was 413±138 ng/mL at 7 hours post implantation. The plasma levels declined over the remainder of the study. The plasma levels apparently reached steady state between Day 70 and Day 91 of the study (61.5±6.1 and 61.5±17.6 ng/mL, respectively). The decrease in plasma nevirapine over time may also have resulted from repositioning/migration of the test implants. At the time of necropsy, the test implants had assumed new configurations relative to each other. The repositioning may have physically impaired the release of nevirapine from the ends of the implants.

### EXAMPLE 2

Summary

The primary objective of this study was to evaluate plasma levels of nevirapine (a non-nucleoside reverse transcriptase inhibitor with activity against Human Immunodeficiency Virus Type 1 [HIV-1]) in female Sprague-Dawley rats following subcutaneous implantation of a test device. This device contained 50 mg of nevirapine and was designed to deliver 0.3 mg nevirapine/day following subcutaneous implantation. Toxicity was assessed through evaluation of clinical observations, body weights, clinical pathology (hematology and serum chemistry), and anatomic pathology of the implant site.

This study consisted of 12 female Sprague-Dawley rats (Group 1); all rats underwent surgical implantation of the test device on Day 1. An additional “sham” rat underwent the same surgical procedures but did not receive an implanted test device. At protocol specified time points, blood was collected and, after it was processed for plasma, was analyzed for nevirapine concentrations by the Test Facility's Bioanalytical Chemistry (BAC) Department. Plasma from the “sham” rat was collected to evaluate the possibility that anesthetics used in surgery might interfere with nevirapine analyses at the early time points. This rat was sacrificed after the 7-hour time point. The other surviving rats were sacrificed on Day 84 after terminal blood samples were obtained for nevirapine bioanalysis, hematology, and serum chemistry.

Plasma nevirapine concentrations remained below the quantitation limit (20 ng/mL) in seven of twelve rats one hour after surgical implantation of the device. The highest plasma concentration among the five other rats one hour after implantation was 26.7 ng/mL. By three hours after implantation, all the sampled rats had detectable levels of nevirapine in plasma; the mean concentration was 100.5 ng/mL. The peak mean plasma nevirapine concentration (322.8 ng/mL) was obtained 12 hours after test device implantation. Mean plasma nevirapine concentrations remained above 200 ng/mL three days after surgery and had decreased to 109.7 ng/mL on Day 7. On subsequent days, mean plasma nevirapine concentrations remained below 100 ng/mL and ranged from approximately 30-80 ng/mL during the remaining portion of the study.

Introduction

The objective of this study was to determine the plasma concentrations and toxicity of nevirapine following subcutaneous implantation in rats.

Experimental Design

Overview

The study consisted of one group of 12 female Sprague-Dawley rats. On Day 1, surgical implantation of the test device, nevirapine implant (50 mg), into the dorsal thoracolumbar region was performed. The implants were designed to release 0.3 mg of nevirapine per day. At protocol-specified time points, clinical observations were performed and body weights were recorded. Blood samples were collected for analysis of clinical pathology parameters (hematology and serum chemistry) and toxicokinetics. Surviving animals were euthanized on Day 84. Comprehensive necropsy, limited tissue collection, and limited histology were performed.

Study Design

Text Table 1 summarizes the study design.

TEXT TABLE 1
Study Design

| | | | Total | Daily Dose | | |
|---|---|---|---|---|---|---|
| Group | Number of | | Implanted | Level | Dosing | Necropsy |
| Number | Females | Test Device | Dose (mg) | (mg/day) | Regimen | Day |
| 1 | 12 | Nevirapine | 50 | 0.3 | Subcutaneous | 84 |
| | | Implant | | | implantation | |
| | | | | | on Day 1 | |

Materials and Methods

Preoperative Procedures

Anesthesia and Antibiotic Therapy: The animals were pre-anesthetized with atropine SO₄ (0.4 mg/kg, SC). Approximately 10-30 minutes later, the animals were anesthetized with a combination of ketamine/medetomidine (60 mg/kg and 0.3 mg/kg, respectively, IM). Drugs for appropriate anesthetic management were available for administration if indicated. The drug, dose, route, and site of administration were documented in the surgical records.

Surgical Preparation: An ophthalmic ointment was administered to each eye. The fur was removed from the dorsal thoraco-lumbar region, extending laterally on both sides to the lateral midline. Any excess fur was brushed or vacuumed off. The animal was placed in ventral recumbency on a circulating hot water pad in order to help maintain body heat. The surgical area was then gently wiped with 70% isopropyl alcohol, which was allowed to dry. DuraPrep™, or similar solution, was then applied to the area and also allowed to dry.

Surgical Procedures

A 1-2 cm incision was made in the skin over the thoraco-lumbar area, slightly lateral to the dorsal midline on either side of the animal (surgeon preference). A subcutaneous pocket was made under the skin extending ventrally to the level of the panniculus carnosus, thus ensuring adequate blood supply to the overlying skin. The implant was placed in this pocket, and the wound was closed in one layer with appropriately sized absorbable suture material placed in a continuous pattern. The skin was closed with autoclips. The autoclips were removed seven to ten days after surgery. One additional rat (No. 13) underwent a sham surgical procedure that was identical to that described above except that no implant was placed.

Postoperative Care

Recovery: The animals were given atipamezole (1 mg/kg, SC) to reverse the ketamine/medetomidine anesthesia. Animal Nos. 4, 11, 12, and 13 were given two doses of atipamezole (1 mg/kg/dose, SC).

Analgesia Therapy: After recovery from anesthesia, the animals were given an injection of buprenorphine (0.05 mg/kg, SC).

Observations

Moribundity/mortality checks were performed and recorded twice daily for mortality and moribundity. Clinical observations were performed and recorded once weekly beginning on Day 2. Clinical observations included but were not limited to changes in the skin and hair, eyes and mucous membranes, respiratory system, circulatory system, central nervous system, somatomotor activity, and behavior pattern, and the occurrence of tremors, convulsions, salivation, diarrhea, or lethargy.

Body Weights

For the original animals on the study (Animal Nos. 2-12), body weights were recorded on Days-7, 1, 3, 7, 14, 21, 28, 35, 42, 49, 56, 63, 70, 77, and 83. Body weights for surviving replacement animals were measured on Days 1, 7, 14, 21, 28, 35, 42, 49, 56, 63, 70, and 77. A final fasted body weight was obtained on the two surviving replacement animals on Day 84 (Animal Nos. 16 and 17). Body weights were taken prior to the collection of blood on all blood collection days.

Sample Collection

Blood: Blood samples were collected according to the schedule presented in Text Table 2. All toxicokinetic samples (including Day 84) were collected by puncture of the retro-orbital sinus/plexus after the animals were anesthetized with CO₂ excepting the one hour time point samples, which were collected while the animals were still affected by the anesthetic agents used during surgery. Prior to necropsy, blood was collected for clinical pathology by puncture of the abdominal aorta/vena cava after the animals were anesthetized with a ketamine:xylazine:acepromazine mixture. Volumes represent whole blood and are approximate amounts.

TEXT TABLE 2
Sample Collection Schedule

| | | Clinical Pathology | | |
|---|---|---|---|---|
| | Number of | | Serum | BAC |
| Time Point | Animals | Hematology | Chemistry | Toxicokinetics |
| Day-7 | 15 | | | X |
| Day 1 at 1, 3, 7, | 13^{a,b} | | | X |
| 12, and 24 | | | | |
| hours post | | | | |
| implantation | | | | |
| Days 3, 7, 14, | 12 or | | | X |
| 28, 42, 56, | surviving | | | |
| and 70 | animals | | | |
| Day 84 | 12 or | X | X | X |
| | surviving | | | |
| | animals | | | |

| Volume of Whole Blood/ | 1.3 mL | 1.8 mL | 0.75/1.5 mL/ |
|---|---|---|---|
| Time Point | | | animal^{c} |
| Anticoagulant | EDTA | None | EDTA |
| ^{a}All 12 rats were bled at the 1-hour time point. Six rats were bled at 3 and 12 hours. The other six rats were bled at the 7- and 24-hour time points. | | | |
| ^{b}An additional rat received anesthesia, a surgical incision, a subcutaneous pocket, closure of the wound, and reversal of anesthesia but did not receive an implant. These procedures were conducted to mimic the actual timing of the surgical procedure. Blood samples (0.75 mL) were | | | |
| #collected approximately 1, 3, and 7 hours after implantation would have occurred. The blood as processed for plasma, which was used as a control during the bioanalytical phase of the study. The animal was euthanized fol | | | |

^{c}From Day −7 to Day 7, and on Day 70 (replacement animals only) and Day 84, 0.75 mL was collected per animal. From Day 14 to Day 70, 1.5 mL was collected per animal.

Clinical Pathology

Hematology: Blood samples were analyzed for the parameters specified in Text Table 3 using a Bayer ADVIA 120 hematology analyzer.

Text TABLE 3
Hematology Parameters

Total leukocyte count (WBC)
Erythrocyte count (RBC)
Hemoglobin concentration (HGB)
Hematocrit value (HCT)^{a}
Mean corpuscular volume (MCV)
Mean corpuscular hemoglobin (MCH)^{a}
Mean corpuscular hemoglobin concentration (MCHC)^{a}
Platelet count (PLT)
Relative and absolute reticulocyte count (RTC, ARTC)
WBC Differential
Relative and absolute polymorphonuclear neutrophil count (PLY,
APLY)
Relative and absolute lymphocyte count (LYM, ALYM)
Relative and absolute monocyte count (MNO, AMNO)
Relative and absolute eosinophil count (EOS, AEOS)
Relative and absolute basophil count (BSO, ABSO)
Relative and absolute large unstained cell count (LUC, ALUC)
^{a}Calculated value; additionally, all absolute values are calculated.

Serum Chemistry: Blood samples were processed and the parameters specified in Text Table 4 were determined using a Boehringer Mannheim Hitachi 717 chemistry analyzer.

TEXT TABLE 4
Serum Chemistry Parameters

| | | |
|---|---|---|
| Glucose (GLU) | Creatinine (CRE) | Total bilirubin (TBIL) |
| Urea nitrogen (BUN) | Calcium (CAL) | Triglycerides (TRG) |
| Total protein (TPR) | Phosphorus (PHOS) | Alanine aminotrans- |
| | | ferase (ALT) |
| Albumin (ALB) | Sodium (NA) | Aspartate aminotrans- |
| | | ferase (AST) |
| Globulin (GLOB)^{a} | Potassium (K) | Alkaline phosphatase |
| | | (ALK) |
| Albumin/Globulin ratio | Chloride (CL) | Gamma- |
| (A/G)^{a} | Total cholesterol | glutamyltransferase |
| | (CHOL) | (GGT) |

^{a}Calculated value.

Blood for Bioanalytical Chemistry: The blood samples were centrifuged, the plasma was extracted and placed in a ≦−70° C. freezer. Plasma samples were analyzed by a method validated by the Test Facility under a separate protocol.

Pathology

Euthanasia: The animals were euthanized on Day 84 (anesthesia by ketamine:xylazine:acepromazine mixture followed by exsanguination).

Necropsy: A comprehensive necropsy, defined as the macroscopic examination of the external surface of the body, all orifices, and the cranial, thoracic, and abdominal cavities and their contents, was performed on all animals. The implant sites (with underlying muscle layers) including the diameter of the implant plus a few millimeters of surrounding tissue and any gross macroscopic lesions were examined in situ, dissected free, and fixed in 10% neutral buffered formalin. The implants were retrieved, gently cleaned of adhering tissue, and stored dry and frozen at −20° C. until shipped to the Sponsor. Observations noted at necropsy were recorded.

Histology: The implant site and any gross macroscopic lesions were trimmed, embedded, sectioned, and mounted on glass slides. Slides were stained with hematoxylin and eosin.

Results

Surgical Implantation

The test device was surgically implanted successfully in all animals with the exception of the sham (No. 13). The sham rat had the same surgical procedures without actual placement of the test device.

Mortality

Four rats that were implanted on Jul. 31, 2001, died due to CO₂ asphyxiation during a post-operative procedure (wound autoclipping). Four replacement animals were assigned to the study. Hence, the study consisted of eight original animals and four replacements. Of these animals, eight survived until their scheduled euthanasia date (Day 84). Two rats were euthanized (Days 31 and 50) because the test device had begun to exteriorize (i.e., it started to protrude through the skin). Per Test Facility Standard Operating Procedure, these animals were classified as having undergone moribund euthanasia. Two other rats died (Days 28 and 57) following blood collection under CO₂ anesthesia.

The only two deaths associated with the test device occurred in the rats that were euthanized because the device was partially extruded from the implant site.

Plasma Nevirapine Concentrations

The concentration analysis results are summarized in Text Table 5.

Plasma nevirapine concentrations remained below the quantitation limit (20 ng/mL) in seven of twelve rats one hour after surgical implantation of the device. The highest plasma concentration among the five other rats one hour after implantation was 26.7 ng/mL. By three hours after implantation, all the sampled rats had detectable levels of nevirapine in plasma; the mean concentration was 100.5 ng/mL. The peak mean plasma nevirapine concentration (322.8 ng/mL) occurred 12 hours after test device implantation. Mean plasma nevirapine concentrations remained above 200 ng/mL three days after surgery and had decreased to 109.7 ng/mL on Day 7. On subsequent days, mean plasma nevirapine concentrations remained below 100 ng/mL and ranged from approximately 30-80 ng/mL during the remaining portion of the study.

TEXT TABLE 5
Chronological Plasma Nevirapine Concentrations (ng/mL)^{a}

| | Time | | | | | | | | | | | | | | |
|---|---|---|---|---|---|---|---|---|---|---|---|---|---|---|---|
| | after | | | | | | | | | | | | | | |
| | Dosing | Animal Number | | | | | | | | | | | | | |
| Day | (hr) | 2 | 3 | 4 | 6 | 7 | 10 | 11 | 12 | 14 | 15 | 16 | 17 | Mean | SD |
| −7 | Pre | BQL | BQL | BQL | BQL | BQL | BQL | BQL | BQL | BQL | BQL | N/A | N/A | N/A | N/A |
| 1 | 1 | BQL | BQL | BQL | 26.3 | BQL | BQL | BQL | 26.7 | 23.1 | BQL | 21.7 | 21.7 | N/A | N/A |
| | 3 | 78.8 | 91.4 | 93.6 | 152 | N/A | N/A | N/A | N/A | 116 | 70.9 | N/A | N/A | 100/5 | 29.6 |
| | 7 | N/A | N/A | N/A | N/A | 246 | 215 | 104 | 492 | N/A | N/A | 184 | 234 | 245.8 | 130.9 |
| | 12 | 390 | 388 | 318 | 447 | N/A | N/A | N/A | N/A | 246 | 148 | N/A | N/A | 322.8 | 110.2 |
| 2 | 24 | N/A | N/A | N/A | N/A | 351 | 281 | 368 | 390 | N/A | N/A | 119 | 120 | 271.5 | 123.3 |
| 3 | | 199 | 226 | 164 | 225 | 233 | 291 | 268 | 242 | 268 | 216 | 85.5 | 129 | 212.2 | 60.0 |
| 7 | | 124 | 82.1 | 201 | 110 | 119 | 129 | 112 | 100 | 88.7 | 89 | 99.6 | 62.1 | 109.7 | 34.5 |
| 14 | | 133 | 58.4 | 107 | 83.8 | 68.2 | 66.4 | 82.5 | 62.3 | 56.9 | 104 | 51.4 | 65.4 | 78.3 | 24.8 |
| 28 | | 48 | 56.7 | 166 | 52.9 | 51.9 | 50.4 | 40.4 | 39.2 | 63 | 99.3 | 42.9 | 44.3 | 62.9 | 36.2 |
| 42 | | 43.6 | NR | 40.4 | 45 | 43.3 | 47.7 | 37.3 | N/A | 42.6 | N/A | 49 | 44 | 43.7 | 3.5 |
| 56^{b} | | 33.9 | 49.2 | N/A | 29.5 | 37.6 | 54.5 | 34.8 | N/A | N/A | N/A | 38.5 | 32.3 | 38.8 | 8.7 |
| 70 | | 32.5 | 36.7 | N/A | 37 | 38.9 | 39.8 | 29.2 | N/A | N/A | N/A | 29.2 | 28.2 | 33.9 | 4.7 |
| 84 | | 32.3 | 50 | N/A | 43.4 | 45.9 | 53.3 | 42.2 | N/A | N/A | N/A | 25.9 | 39.5 | 41.6 | 9.0 |

BQL = Below quantitation limit;
N/A = Not applicable
^{a}Pre-study (Day −7) plasma nevirapine concentrations for replaced animals (Nos. 1, 5, 8, and 9) and the sham surgery animal (No. 13) were BQL. Plasma nevirapine concentrations for Animal No. 13 remained BQL at 1, 3, and 7 hours post surgery.
^{b}Data presented for Animal Nos. 16 and 17 were generated from samples collected on Day 57.

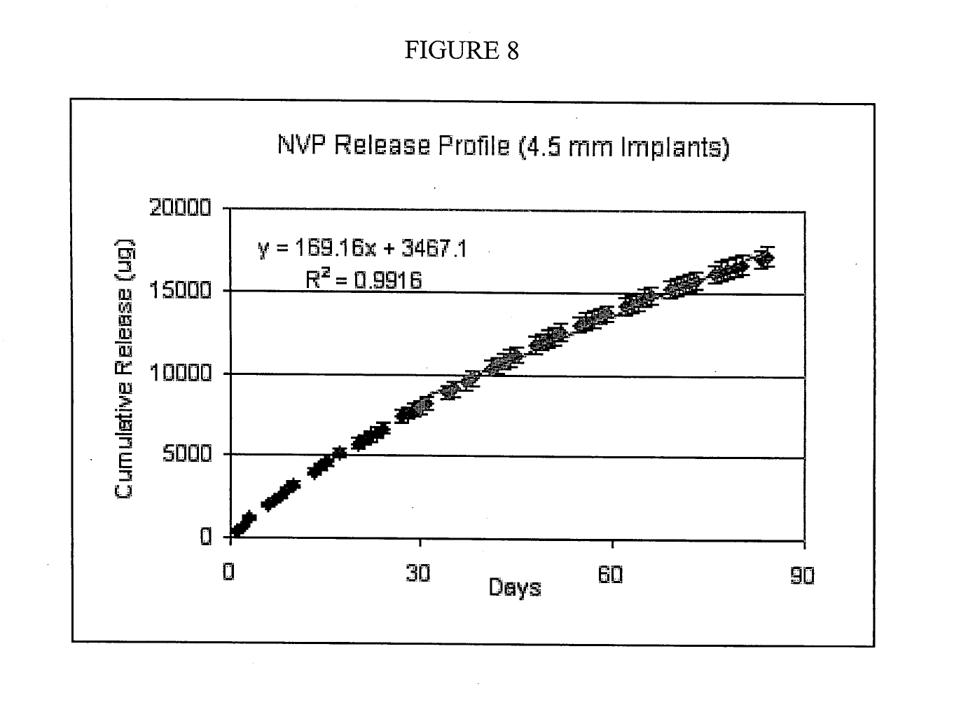

FIG. 8 shows the in vitro release profile of the 4.5 mm NVP implant in 0.1M phosphate buffer (pH 7.4) at 37° C.

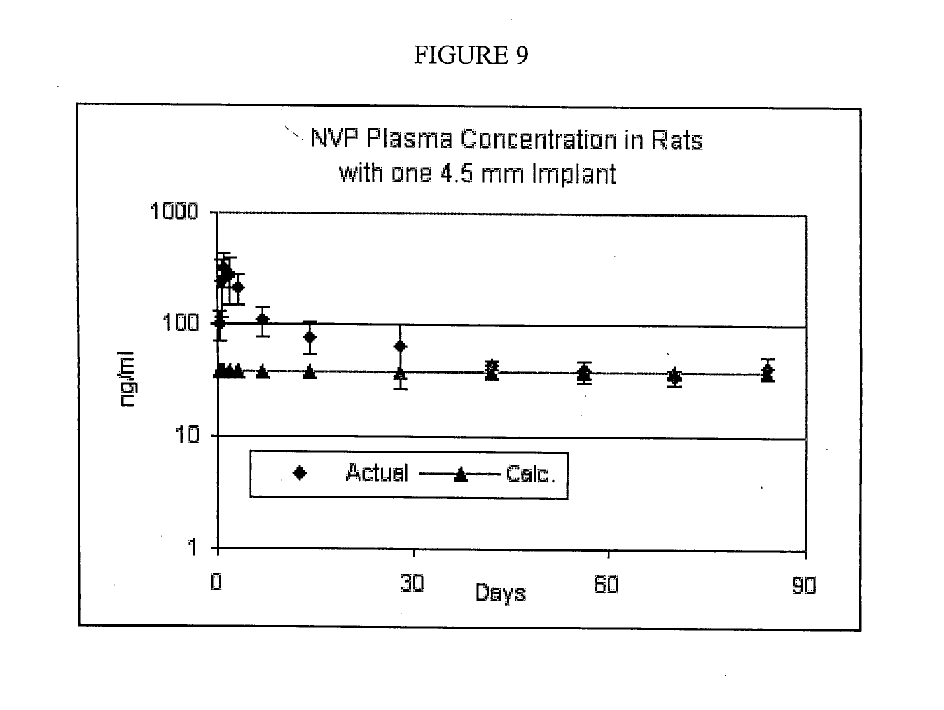

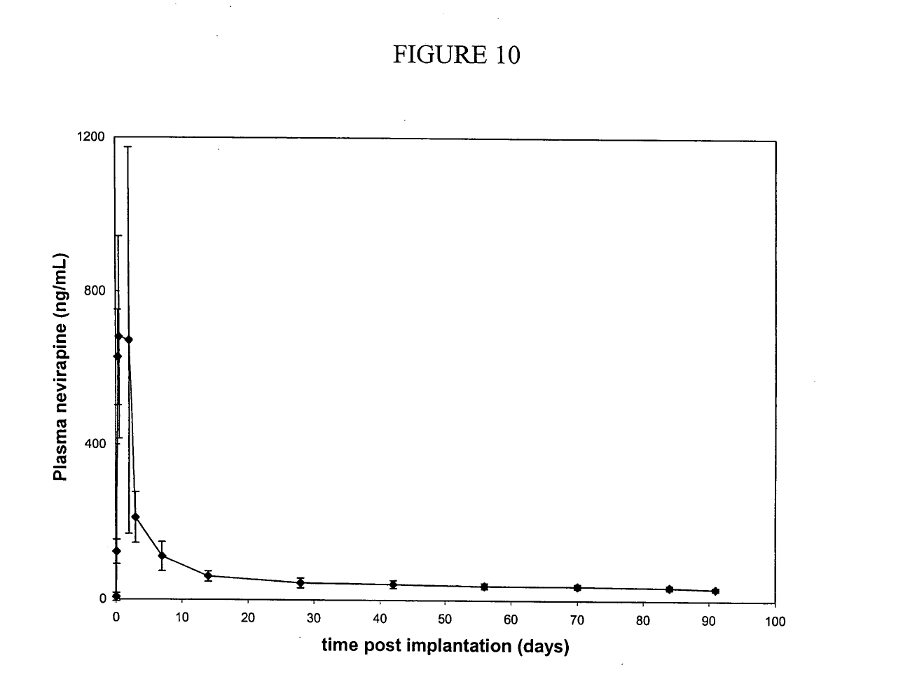

FIG. 9 shows the NVP plasma concentration, from table above, in rats (line marked with diamonds) with one 4.5 mm implant surgically inserted subcutaneously, in comparison with the calculated NVP plasma level (line marked with triangles).

Calculation of Nevirapine (NVP) Plasma Concentration in Rats:

Based on the in vitro release rate (kᵣ), animal body weight (W, 300 gm) and known NVP PK-data [apparent volume of distribution (Vₛₛ): 984 ml/kg and elimination constant (kₑₗ): 0.629 hr⁻¹ in rats] and assuming that the PK follows a one-compartment model, the NVP plasma concentration (C) in rats at steady state can be calculated using the following equation:

C=kᵣ/(kₑₗWVₛₛ)

With an in vitro release rate of 169 ug/day (see FIG. 8) for the NVP implant, at steady state, a NVP plasma concentration of 38 ng/ml is expected for rats with six 2.0 mm rod implants having releasing ports on shell. The calculated NVP concentrations are displayed in FIG. 9 (line marked with triangle).

Clinical Pathology

Hematology: Thee mean total white blood cell count (WBC) on Day 84 for the implanted rats (2.8×10³ cells/μL) was lower than the published normal range (5-14×10³ cells/μL) for female Sprague-Dawley rats. In addition, it was lower than mean WBC values obtained for control female Sprague-Dawley rats in three other recently conducted in-house studies (range=5.2-10.8×10³ cells/μL). Hence, it appears that leukopenia was associated with implantation of the nevirapine-containing test device. The reduction in total WBC mirrored a reduction in the mean absolute lymphocyte count (ALYM) (1.9×10³ cells/μL) as compared to mean in-house values for control female Sprague-Dawley rats (4.4-9.3×10³ cells/μL) from three previous studies. This difference is even more striking when the mean relative lymphocyte count (LYM) in the present study (66.1%) is compared to that of control female Sprague-Dawley rats from the aforementioned three previous in-house studies (82-86%). In summary, implantation of the test device appeared to be associated with an overall reduction in white blood cell counts with lymphocytes showing the greatest reduction.

Serum Chemistry: Without baseline pre-treatment values, the mean alanine aminotransferase (ALT) (59.7 U/L) and aspartate aminotransferase levels (AST) (141.4 U/L) on Day 84 in this study are difficult to interpret. Although both means are higher than published values (ALT equals 10-50 U/L and AST equals 45-100 U/L), both remain within the range exhibited by control female Sprague-Dawley rats recently tested in-house in three other studies (ALT range equals 34.0-123 U/L and AST range equals 98-285 U/L). With regards to these enzymes, there also appeared to be a highly responsive animal (No. 7) relative to the others. The mean blood urea nitrogen (BUN) level in the present study (25.3 mg/dL) was apparently elevated relative to the controls from the three in-house studies (13.6-18.5 mg/dL) and was also higher than published values for this species (12-20 mg/dL). This may suggest an effect of nevirapine on renal function however, another indicator of possible nephrotoxicity, mean serum creatinine (CRE) (0.67 mg/dL), was only slightly outside of the range exhibited by control females in other in-house studies (0.46-0.60 mg/dL) and was within the published normal range for Sprague-Dawley rats (0.3-0.9 mg/dL). In summary, implantation of the nevirapine-containing test device was not strongly associated with any apparent adverse effects upon serum chemistry.

Conclusion

The primary objective of this study was to evaluate plasma levels of nevirapine in female Sprague-Dawley rats following subcutaneous implantation of a test device. This device contained 50 mg of nevirapine and was designed to deliver 0.3 mg nevirapine/day following subcutaneous implantation. Toxicity was assessed through evaluation of clinical observations, body weights, clinical pathology (hematology and serum chemistry), and anatomic pathology of the implant site.

Plasma nevirapine concentrations remained below the quantitation limit (20 ng/mL) in seven of twelve rats one hour after surgical implantation of the device. The highest plasma concentration among the five other rats one hour after implantation was 26.7 ng/mL. By three hours after implantation, all the sampled rats had detectable levels of nevirapine in plasma; the mean concentration was 100.5 ng/mL. The peak mean plasma nevirapine concentration (322.8 ng/mL) was obtained 12 hours after test device implantation. Mean plasma nevirapine concentrations remained above 200 ng/mL three days after surgery and had decreased to 109.7 ng/mL on Day 7. On subsequent days, mean plasma nevirapine concentrations remained below 100 ng/mL and approximated 30-80 ng/mL during the remaining portion of the study.

Post-surgery body weights in nine of twelve rats did not return to pre-surgery (Day 1) levels until Day 14 or afterwards. Nevirapine may have affected weight gain in these animals but corresponding data with sham-operated rats was not available for direct comparison. Leukopenia, mainly as a result of a decrease in the number of circulating lymphocytes, was associated with implantation of the test device.

The no-observable-adverse-effects level (NOAEL) for the subcutaneously implanted nevirapine-containing device could not be determined for female Sprague-Dawley rats in this study.

### EXAMPLE 3

Summary

The primary objective of this study was to evaluate plasma levels of nevirapine (a non-nucleoside reverse transcriptase inhibitor with activity against Human Immunodeficiency Virus Type 1 [HIV-1]) in female Sprague-Dawley rats, following subcutaneous implantation of a test implant. Each test implant contained 47.4 mg nevirapine, 2.5 mg polyvinyl alcohol (PVA), and 0.1 mg magnesium stearate and was dip-coated in PVA before being inserted into a precut silicone tube. The silicone tube included several ports to allow passage of nevirapine out of the device. The approximate size of each implant was 1.5 cm (length)×2 mm (diameter). Sham implants consisted of silicone tubing identical to that used to house the test implants. A single implant was surgically placed into a subcutaneous pocket in the scapular region of each rat on Day 1 (Jun. 4, 2003). Two Group 1 rats received the sham implant, and 10 Group 2 rats received the nevirapine test implant. Toxicity was assessed during the study through evaluation of clinical observations, body weights, and macroscopic pathology of the implant site following euthanasia on Day 91 (Sep. 2, 2003). Blood samples were collected at specified time points during the study for analysis of plasma nevirapine concentrations.

The most common clinical observation was alopecia, mainly of the limbs or abdomen. This finding was found in animals from both groups. Other findings that were of low incidence included scab, chromodacryorrhea, opaque eye, rough hair coat, and skin erythema. Mean body weight values for the sham and test implant (Groups 1 and 2) rats increased over time in a similar manner. Macroscopic necropsy findings were limited to opaque/dry foci on the eyes of two Group 2 rats.

Blood samples were collected on Day 1 at 1, 3, 7, 12, and 24 hours after implantation and then on Days 3, 7, 14, 28, 42, 56, 70, 84 and 91. Nevirapine plasma concentration analysis of these samples showed that initially three of ten animals had low plasma levels (just over 20 ng/mL) at 1 hour after implantation. The results for the remaining animals at 1 hour after implantation were BQL (below the quantitation limit) of 20 ng/mL. The mean plasma levels rose to 123 ng/mL at 3 hours after implantation with all five animals bled at that time point showing detectable levels. Over the next three time points mean plasma levels were 627.6±124.90 ng/mL, 680.2±264.03 ng/mL, and 671.8±502.52 ng/mL for 7, 12, and 24 hours after implantation, respectively. Mean plasma levels were 211.6±65.16 ng/mL and 111.3±37.76 ng/mL at Days 3 and 7, respectively. The mean plasma levels declined slowly over the remainder of the study to a low of 30.6±4.87 ng/mL at Day 91.

TEXT TABLE 1
Chronological Plasma Nevirapine Concentrations (ng/mL)

| | Time | | | | | | | | | | | | |
|---|---|---|---|---|---|---|---|---|---|---|---|---|---|
| | after | | | | | | | | | | | | |
| | Dosing | Animal Number^{a} | | | | | | | | | | | |
| Day | (hr) | 5 | 6 | 8 | 9 | 10 | 11 | 12 | 13 | 14 | 15 | Mean | SD^{a} |
| 1 | 1 | 0 | 20.6 | 0 | 0 | 0 | 20.5 | 0 | 22.8 | 0 | 0 | 6.4 | 10.31 |
| | 3 | 82.8 | 117 | 112 | N/A | N/A | N/A | N/A | N/A | 134 | 169 | 123.0 | 31.66 |
| | 7 | N/A | N/A | N/A | 672 | 824 | 522 | 532 | 588 | N/A | N/A | 627.6 | 124.90 |
| | 12 | 487 | 621 | 526 | N/A | N/A | N/A | N/A | N/A | 1140 | 627 | 680.2 | 264.03 |
| 2 | | N/A | N/A | N/A | 1520 | 744 | 331 | 408 | 356 | N/A | N/A | 671.8 | 502.52 |
| 3 | | 145 | 189 | 133 | 315 | 248 | 131 | 189 | 216 | 276 | 274 | 211.6 | 65.16 |
| 7 | | 53.4 | 100 | | 84.6 | 107 | 98.5 | 107 | 119 | 188 | 144 | 111.3 | 37.76 |
| 14 | | 44.8 | 53.7 | | 41.7 | 63.1 | 65.1 | 66.4 | 61.2 | 87.2 | 57.8 | 60.1 | 13.36 |
| 28 | | 29.3 | 36.5 | | 23.6 | 63.6 | 49.2 | 47.8 | 54.4 | 47.4 | 41.6 | 43.7 | 12.44 |
| 42 | | 28.1 | 40.8 | | 28.7 | 48.8 | 37.0 | 39.3 | 50.1 | 57.6 | 35.7 | 40.7 | 9.89 |
| 56 | | 30.7 | 33.3 | | 23.2 | 46.7 | 35.3 | 43.1 | 40.9 | 39.6 | 40.6 | 37.0 | 7.18 |
| 70 | | 29.1 | 35.8 | | 25.4 | 38.5 | 37.1 | 43.1 | 38.5 | 39.1 | 41.7 | 36.5 | 5.75 |
| 84 | | 30.8 | 35.3 | | 23.2 | 41.1 | 34.5 | 41.5 | 38.4 | 30.2 | 33.2 | 34.2 | 5.80 |
| 91 | | 28.1 | 25.2 | | 24.5 | 39.0 | 31.6 | 33.3 | 32.5 | 34.8 | 26.5 | 30.6 | 4.87 |

N/A = Not applicable because blood samples were not obtained for these time points.
^{a}For the purpose of determining the mean and standard deviation, BQL was set to zero.

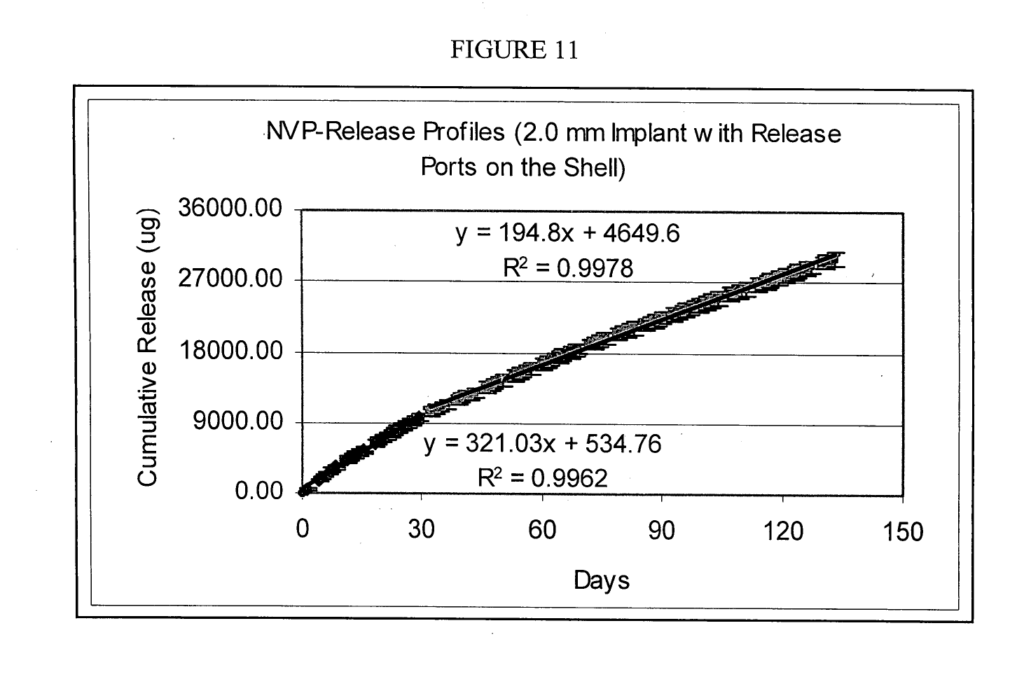

FIG. 11 shows the in vitro release profile of the 2.0 mm NVP implant containing releasing ports on the shell, in 0.1M phosphate buffer (pH 7.4) at 37° C.

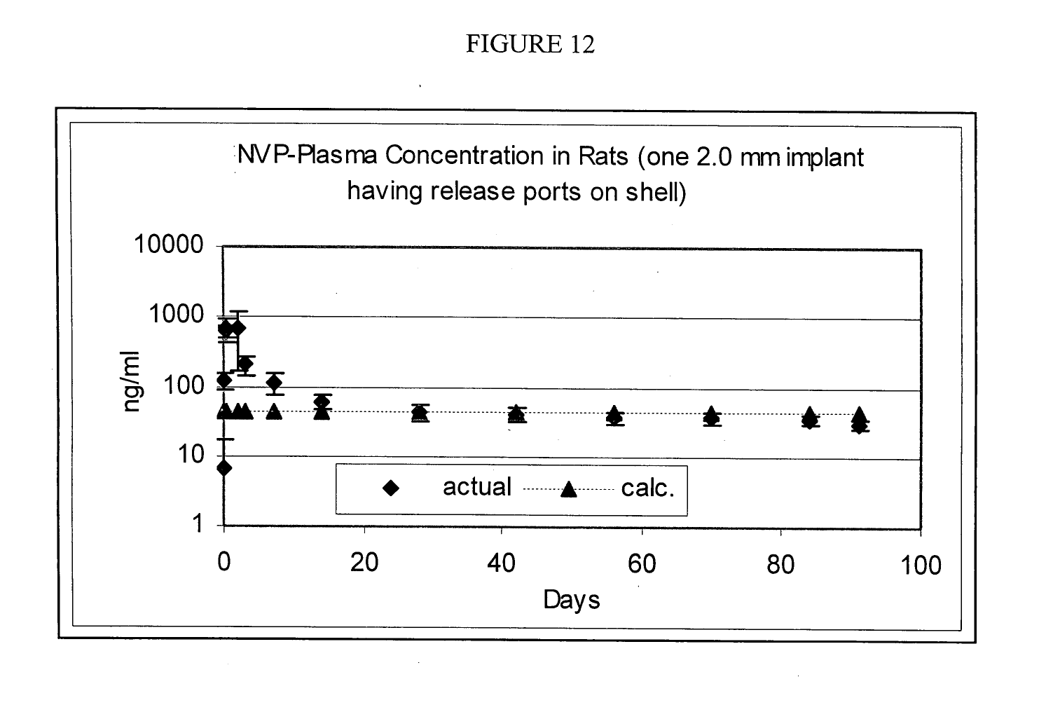

FIG. 12 shows the NVP plasma concentration, from table above, in rats (line marked with diamonds) with one 2.0 mm implant containing releasing ports on the shell, surgically inserted subcutaneously, in comparison with the calculated NVP plasma level (line marked with triangles).

Calculation of Nevirapine (NVP) Plasma Concentration in Rats:

Based on the in vitro release rate (kᵣ), animal body weight (W, 300 gm) and known NVP PK-data [apparent volume of distribution (Vₛₛ): 984 ml/kg and elimination constant (kₑₗ): 0.629 hr⁻¹ in rats] and assuming that the PK follows a one-compartment model, the NVP plasma concentration (C) in rats at steady state can be calculated using the following equation:

C=kᵣ/(kₑₗWVₛₛ)

With an in vitro release rate of 194.8 ug/day (see FIG. 11) for the NVP implant, at asteady state, a NVP plasma concentration of 44 ng/ml is expected for rats with one 2.0 mm implant having releasing ports on the shell. The calculated NVP concentrations are displayed in FIG. 12 (line marked with triangle).

The sham and test devices did not cause any significant abnormalities in the rats under the conditions of this study. Mean nevirapine plasma concentrations increased after implantation with the peak at 12 hours post implantation, but with relatively steady levels present at 7, 12, and 24 hours, before declining after that point (beginning on Day 3).

Study Design

| | | | Total | Daily Dose | | |
|---|---|---|---|---|---|---|
| Group | Number of | | Implanted | Level | Dosing | Necropsy |
| Number | Females | Test Device | Dose (mg) | (μg/day) | Regimen | Day |
| 1 | 2 | Sham Implant | 0 | 0 | Subcutaneous | 91 |
| 2 | 10 | Test Implant | 50 | 300 | implantation | |
| | | | | | on Day 1 | |

Test Device Identification

| | |
|---|---|
| Name: | Test Implants (50 mg nevirapine) |
| Physical Description: | Each implant contains 47.4 mg |
| | nevirapine, 2.5 mg PVA, and 0.1 mg of |
| | magnesium stearate. The implant is dip- |
| | coated in PVA before it is inserted into a |
| | precut silicone tube. The approximate |
| | size of each implant is 1.5 cm (length) × 2 mm |
| | (diameter). |

Control Device:

| | |
|---|---|
| Name: | Sham Implants |
| Physical Description: | Silicone tubing identical to that used to |
| | house the Test Implants |

Frequency and Duration of Administration

Doses were administered continuously via subcutaneous implant in the interscapular region for 90 days. The test implant was designed to release approximately 300 μg of nevirapine per day.

### EXAMPLE 4

### Correlation of In Vitro-In Vivo Release Rates for Sustained Release Nevirapine-Implants in Rats

a. Purpose

Sustained release NVP-implants have been designed and developed for the prevention of maternal transmission in AIDS patients. The purpose of this study was to evaluate the in vitro-in vivo release rate correlation for these implants using rats.

b. Methods

Nevirapine was mixed with 5% polyvinyl alcohol (PVA) solution and granulated. Rod-shaped NVP pellets (2.0 mm or 4.5 mm in diameter) were hand compressed using the granules. The pellets were dip coated in 5% PVA solution, air-dried, and inserted into precut silicone tubes. The entire assembly (Implant) was coated in 5% PVA solution and air-dried followed by heat treatment. After gamma-irradiation, in vitro release testing was conducted using 0.1 M phosphate buffer (pH 7.4) at 37° C. as the release medium. The amount of NVP release was determined by HPLC. The sterilized implants (either one 4.5 mm or six 2.0 mm-implants per rat) were implanted subcutaneously in female Sprague-Dawley rats. Blood samples were taken periodically and the plasma concentration of NVP was determined.

c. Results

In vitro NVP was released from the implant in a well-controlled and sustained fashion. Zero-order release profiles were obtained. The 4.5 mm-implant gave a sustained release rate of 169 μg/day, while the 2.0 mm-implant released 52 μg/day for the duration of the test period (over 10 weeks) in vitro. Based on the in vitro release rate, the body weight of rats and known NVP PK-data (distribution volume, kₑₗ) in rats, a plasma concentration of 38 ng/ml or 70 ng/ml was predicted for rats receiving one 4.5 mm-implant or six 2.0 mm-implants respectively. Steady-state plasma concentrations of NVP following subcutaneous implantation were 35˜45 ng/ml and 60˜80 ng/ml.

d. Conclusions

Sustained NVP delivery systems with different release rates were developed. The release rates were determined in vitro in buffer and in vivo in rats. The results indicated that the correlation between in vitro and in vivo release rates was excellent.

From the foregoing description, one of ordinary skill in the art can easily ascertain the essential characteristics of the instant invention, and without departing from the spirit and scope thereof, can make various changes and/or modifications of the invention to adapt it to various usages and conditions. As such, these changes and/or modifications are properly, equitably and intended to be, within the full range of equivalence of the following claims.

What is claimed is:

1. A sustained release drug delivery system comprising
an inner drug core comprising an amount of an antiviral agent,
an inner tube impermeable to the passage of said agent, said inner tube having first and second ends and covering at least a portion of said inner drug core, said inner tube being dimensionally stable,
an impermeable member positioned at said inner tube first end, said impermeable member preventing passage of said agent out of said drug core through said inner tube first end, and
a permeable member positioned at said inner tube second end, said permeable member allowing diffusion of said agent from said drug core through said inner tube second end.

2. A sustained release drug delivery system comprising
a drug core comprising an amount of an antiviral agent,
a first polymer coating permeable to the passage of said agent, and
a second polymer coating impermeable to the passage of said agent,
wherein the second polymer coating covers a portion of the surface area of the drug core and/or the first polymer coating.

3. A sustained release drug delivery system comprising
a drug core comprising an amount of an antiviral agent,
a first polymer coating and a second polymer coating permeable to the passage of said agent,
wherein the two polymer coatings are bioerodible and erode at different rates.

4. A sustained release drug delivery system comprising
a drug core comprising an amount of an antiviral agent,
a first polymer coating permeable to the passage of said agent covering at least a portion of the drug core,
a second polymer coating essentially impermeable to the passage of said agent covering at least a portion of the drug core or the first polymer coating,
and a third polymer coating permeable to the passage of said agent essentially completely covering the drug core and the second polymer coating,
wherein a dose of said agent is released for at least 7 days.

5. A sustained release drug delivery system comprising
a drug core comprising an amount of an antiviral agent,
a first polymer coating permeable to the passage of said agent covering at least a portion of the drug core,
a second polymer coating essentially impermeable to the passage of said agent covering at least a portion of the drug core or the first polymer coating,
and a third polymer coating permeable to the passage of said agent essentially completely covering the drug core and the second polymer coating,
wherein release of said agent maintains a desired concentration of said agent in blood plasma for at least 7 days.

6. A sustained release drug delivery system comprising
a drug core comprising an amount of an antiviral agent, and
a non-erodible polymer coating, the polymer coating being permeable to the passage of said agent covering the drug core and is essentially non-release rate limiting,
wherein a dose of said agent is released for at least 7 days.

7. A sustained release drug delivery system comprising
a drug core comprising an amount of an antiviral agent, and
a non-erodible polymer coating, the polymer coating being permeable to the passage of said agent covering the drug core and is essentially non-release rate limiting,
wherein release of said agent maintains a desired concentration of said agent in blood plasma for at least 7 days.

8. A sustained release drug delivery system comprising
a drug core comprising an amount of an antiviral agent,
a first polymer coating permeable to the passage of said agent covering at least a portion of the drug core,
a second polymer coating essentially impermeable to the passage of said agent covering at least 50% of the drug core and/or the first polymer coating, said second polymer coating comprises an impermeable film and at least one impermeable disc, and
a third polymer coating permeable to the passage of said agent essentially completely covering the drug core, the uncoated portion of the first polymer coating, and the second polymer coating,
wherein a dose of said agent is released for at least 7 days.

9. A sustained release drug delivery system comprising
a drug core comprising an amount of an antiviral agent,
a first polymer coating permeable to the passage of said agent covering at least a portion of the drug core,
a second polymer coating essentially impermeable to the passage of said agent covering at least 50% of the drug core and/or the first polymer coating, said second polymer coating comprises an impermeable film and at least one impermeable disc, and
a third polymer coating permeable to the passage of said agent essentially completely covering the drug core, the uncoated portion of the first polymer coating, and the second polymer coating,
wherein release of said agent maintains a desired concentration of said agent in blood plasma for at least 7 days.

10. A method for treating or reducing the risk of retroviral or lentiviral infection comprising implanting a sustained release drug delivery system including an antiviral agent in a patient in need of treatment wherein a dose of said agent is released for at least 7 days.

11. A method for treating or reducing the risk of retroviral or lentiviral infection comprising implanting a sustained release drug delivery system including an antiviral agent in a patient in need of treatment wherein release of said agent maintains a desired concentration of said agent in blood plasma for at least 7 days.

12. The system according to claim 1, wherein the system reduces the risk of mother to child transmission of viral infections.

13. The system according to claim 1, wherein the system treats or reduces the risk of retroviral or lentiviral infection.

14. The system according to claim 13, wherein the retroviral or lentiviral infections include HIV, Bowenoid Papulosis, Chickenpox, Childhood HIV Disease, Human Cowpox, Hepatitis C, Dengue, Enteroviral, Epidermodysplasia Verruciformis, Erythema Infectiosum (Fifth Disease), Giant Condylomata Acuminata of Buschke and Lowenstein, Hand-Foot-and-Mouth Disease, Herpes Simplex, Herpes Virus 6, Herpes Zoster, Kaposi Varicelliform Eruption, Rubeola Measles, Milker's Nodules, Molluscum Contagiosum, Monkeypox, Orf, Roseola Infantum, Rubella, Smallpox, Viral Hemorrhagic Fevers, Genital Warts, and Nongenital Warts.

15. The system according to claim 1, wherein the antiviral agent is selected from azidouridine, anasmycin, amantadine, bromovinyldeoxusidine, chlorovinyldeoxusidine, cytarbine, didanosine, deoxynojirmycin, dideoxycitidine, dideoxyinosine, dideoxynudeoside, desciclovir, deoxyacyclovir, edoxuidine, enviroxime, fiacitabine, foscamet, fialuridine, fluorothymidine, fluxuridine, hypericin, interferon, interlenkin, isethionate, nevirapine, pentamidine, ribavirin, rimantadine, stavirdine, sargramostin, suramin, trichosanthin, tribromothymidine, trichlorothymidine, vidarabine, zidoviridine, zalcitabine, and 3-azido-3-deoxythymidine, and pharmaceutically acceptable salts, analogs, prodrugs or codrugs thereof.

16. The system according to claim 1, wherein the antiviral agent is selected from nevirapine, delavirdine, and efavirenz, and pharmaceutically acceptable salts, analogs, prodrugs or codrugs thereof.

17. The system according to claim 1, wherein the antiviral agent is nevirapine, or a pharmaceutically acceptable salt, analog, prodrug, or codrug thereof.

18. The system according to claim 1, wherein the antiviral agent is selected from 2′,3′-dideoxyadenosine (ddA), 2′,3′-dideoxyguanosin (ddG), 2′,3′-dideoxycytidine (ddC), 2′,3′-dideoxythymidine (ddT), 2′3′-dideoxy-dideoxythymidine (d4T), 2′-deoxy-3′-thia-cytosine (3TC or lamivudime), 2′,3′-dideoxy-2′-fluroadenosine, 2′,3′-dideoxy-2′-fluoroinosine, 2′,3′-dideoxy-2′-fluorothymidine, 2′,3′-dideoxy-2′-fluorocytosine, 2′3′-dideoxy-2′,3′-didehydro-2′-fluorothymidine (Fd4T), 2′3′-dideoxy-2′-beta-fluoroadenosine (F-ddA), 2′3′-dideoxy-2′-beta-fluoro-inosine (F-ddI), and 2′,3′-dideoxy-2′-beta-flurocytosine (F-ddC), and pharmaceutically acceptable salts, analogs, prodrugs or codrugs thereof.

19. The system according to claim 1, wherein the antiviral agent is selected from trisodium phosphomonoformate, ganciclovir, trifluorothymidine, acyclovir, 3′azido-3′thymidine (AZT), dideoxyinosine (ddI), and idoxuridine, and pharmaceutically acceptable salts, analogs, prodrugs or codrugs thereof.

20. The system according to claim 1, wherein the release of said agent has a systemic effect.

21. The system according to claim 1, wherein the release of said agent has a local effect.

22. The system according to claim 1, wherein the amount or dose of agent released from the drug delivery system may be a therapeutically effective or a sub-therapeutically effective amount.

23. The system according to claim 1, wherein the amount of the agent within the drug core or reservoir is at least 1 mg to about 500 mg.

24. The system according to claim 1, wherein the amount of the agent within the drug core or reservoir is at least about 2 mg to about 15 mg.

25. The system according to claim 1, wherein a therapeutically effective amount or dose of the agent is released for at least two weeks.

26. The system according to claim 1, wherein a therapeutically effective dose is at least about 30 ng/day, 100 ng/day, or 100 ng/day.

27. The system according to claim 1, wherein the desired concentration of said agent in blood plasma is about 20-100 ng/ml.

28. The system according to claim 1, wherein the system is between about 1 to 30 mm in length.

29. The system according to claim 1, wherein the system is between about 0.5 to 5 mm in diameter.

30. The system according to claim 1, wherein the permeable member comprises a material selected from cross-linked polyvinyl alcohol, polyolefins, polyvinyl chlorides, cross-linked gelatins, insoluble and nonerodible cellulose, acylated cellulose, esterified celluloses, cellulose acetate propionate, cellulose acetate butyrate, cellulose acetate phthalate, cellulose acetate diethyl-aminoacetate, polyurethanes, polycarbonates, and microporous polymers formed by co-precipitation of a polycation and a polyanion modified insoluble collagen.

31. The system according to claim 1, wherein the permeable member comprises cross-linked polyvinyl alcohol.

32. The system according to claim 1, wherein the impermeable member comprises a material selected from polyvinyl acetate, cross-linked polyvinyl butyrate, ethylene ethylacrylate copolymer, polyethyl hexylacrylate, polyvinyl chloride, polyvinyl acetals, plasiticized ethylene vinylacetate copolymer, polyvinyl acetate, ethylene vinylchloride copolymer, polyvinyl esters, polyvinylbutyrate, polyvinylformal, polyamides, polymethylmethacrylate, polybutylmethacrylate, plasticized polyvinyl chloride, plasticized nylon, plasticized soft nylon, plasticized polyethylene terephthalate, natural rubber, polyisoprene, polyisobutylene, polybutadiene, polyethylene, polytetrafluoroethylene, polyvinylidene chloride, polyacrylonitrile, cross-linked polyvinylpyrrolidone, polytrifluorochloroethylene, chlorinated polyethylene, poly(1,4′-isopropylidene diphenylene carbonate), vinylidene chloride, acrylonitrile copolymer, vinyl chloride-diethyl fumerale copolymer, silicone rubbers, medical grade polydimethylsiloxanes, ethylene-propylene rubber, silicone-carbonate copolymers, vinylidene chloride-vinyl chloride copolymer, vinyl chloride-acrylonitrile copolymer and vinylidene chloride-acrylonitride copolymer.

33. The system according to claim 32, wherein the impermeable member or the inner tube comprises silicone.

34. The system according to claim 32, wherein the impermeable member is a tube.

35. The system according to claim 32, wherein the tube includes one or more pores.

36. The system according to claim 1, wherein the drug core comprises a pharmaceutically acceptable carrier.

37. The system according to claim 1, wherein the drug core comprises 0.1 to 100% drug, 0.1 to 10% magnesium stearate, and 0.1 to 10% polyethylene glycol.

38. A pharmaceutical package including one or more antiviral compounds formulated for sustained release, and associated with instructions or a label for use in infants who are at risk of maternal transmission of virus.
